(19) United States
(12) Patent Application Publication
(10) Pub. No.: US 2024/0336626 A1
(43) Pub. Date: Oct. 10, 2024

(54) ORGANIC COMPOUND AND USE THEREOF

(71) Applicants: Wuhan Tianma Microelectronics Co., Ltd. Shanghai Branch, Shanghai (CN); Wuhan Tianma Microelectronics Co., Ltd., Wuhan (CN)

(72) Inventors: Ying LIU, Shanghai (CN); Yupei ZHANG, Shanghai (CN); Dongyang DENG, Shanghai (CN)

(73) Assignees: Wuhan Tianma Microelectronics Co., Ltd. Shanghai Branch, Shanghai (CN); Wuhan Tianma Microelectronics Co., Ltd., Wuhan (CN)

(21) Appl. No.: 18/738,508

(22) Filed: Jun. 10, 2024

(30) Foreign Application Priority Data
Feb. 22, 2024 (CN) 202410200841.0

(51) Int. Cl. C07D 498/04; C07D 401/04; C09K 11/02; C09K 11/06; H10K 85/60

(52) U.S. Cl. CPC C07D 498/04; C07D 401/04; C09K 11/02; C09K 11/06; H10K 85/622; H10K 85/631; H10K 85/654; H10K 85/6572

(57) ABSTRACT

Provided are an organic compound and use thereof. Specifically, an organic compound, an OLED display panel, and an electronic device are provided. The organic compound has a structure shown in Formula (I); the OLED display panel comprises a first electrode, a second electrode, and an organic thin film layer provided between the first electrode and the second electrode; the organic thin film layer comprises a light-emitting layer; and a host material of the light-emitting layer comprises any one or a combination of at least two of the organic compounds; and the electronic device comprises the OLED display panel. The compound, when used as a host material of the light-emitting layer for the preparation of an OLED device, can reduce the driving voltage of the device, and improve the luminous efficacy and lifetime of the device.

### CROSS-REFERENCE TO RELATED APPLICATION(S)

This application claims priority to Chinese Patent Application No. 202410200841.0 filed Feb. 22, 2024, the disclosure of which is incorporated herein by reference in its entirety.

### TECHNICAL FIELD

The present application belongs to the technical field of organic electroluminescent materials, relates to an organic compound and use thereof, and especially relates to an organic compound, an organic light-emitting diode display panel, and an electronic device.

### BACKGROUND

As a new generation of display technology, organic electroluminescent materials, also known as organic light-emitting diode (OLED), has the advantages of ultra-thin, self-luminous, wide viewing angle, fast response, high luminous efficacy, good temperature adaptability, simple manufacture process, low driving voltage, low power consumption, etc., and it has been widely used in industries such as flat panel displays, flexible displays, solid-state lighting, and in-vehicle displays.

The organic electroluminescent materials can be divided into electrofluorescence and electrophosphorescence according to a luminescence mechanism, where the electrofluorescence is the radiative decay and transition of singlet excitons while the electrophosphorescence is light emitted during the radiative decay of triplet excitons to a ground state. According to the theory of spin quantum statistics, singlet excitons and triplet excitons are formed at a ratio of 1:3. An electrofluorescent material has an internal quantum efficiency lower than or equal to 25% and an external quantum efficiency which is generally lower than 5%. An electrophosphorescent material has an internal quantum efficiency of 100% in theory and an external quantum efficiency which can reach 20%. In 1998, Prof Ma Yuguang of Jilin University, China, and Prof. Forrest of Princeton University, USA, reported that an osmium complex and a platinum complex were doped as dyes into a light-emitting layer separately. They have successfully discovered and explained the phenomenon of phosphorescent electroluminescence for the first time and pioneered the application of the prepared phosphorescent material to an organic electroluminescent device.

Since a phosphorescent heavy metal material has a relatively long lifetime which can reach the level of μs, the phosphorescent heavy metal material may cause triplet-triplet annihilation and concentration quenching at a high current density, resulting in the degradation of device performance. Therefore, the phosphorescent heavy metal material is generally doped into a suitable host material to form a host-guest doping system, so as to optimize energy transfer and maximize luminescence efficiency and a lifetime. In the current research, the commercialization of heavy metal doping materials is mature and it is difficult to develop alternative doping materials. Therefore, it is a common idea among researchers to focus on the development of phosphorescent host materials.

However, the existing light-emitting host materials suffer from inadequate lifetime, insufficient efficiency, and relatively high driving voltage.

Therefore, it is expected to develop a series of novel, high-performance light-emitting host materials in this field, which can improve the efficiency and lifetime of OLED devices and reduce the driving voltage when used in OLED devices.

### SUMMARY

The present application is to provide an organic compound and use thereof, and especially an organic compound, an OLED display panel, and an electronic device.

In a first aspect, the present application provides an organic compound, and the organic compound has a structure represented by Formula (I):

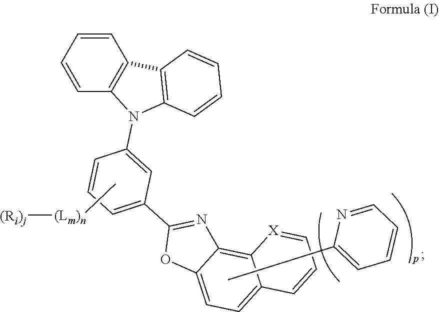

in formula (I), Lₘ is independently selected from any one of a single bond, substituted or unsubstituted C6-C40 arylene, or substituted or unsubstituted C5-C40 heteroarylene;
in formula (I), n is an integer selected from 1 to 4, and m is an integer selected from 1 to n;
in formula (I), Rᵢ is independently selected from any one of substituted or unsubstituted C6-C60 aryl, substituted or unsubstituted C6-C60 heteroaryl, substituted or unsubstituted C6-C60 fused ring aryl, or substituted or unsubstituted C6-C30 arylamino;
in formula (I), j is an integer selected from 1 to 4, and i is an integer selected from 1 to j; and j and n have the same value;
in formula (I), X is selected from N or C—R_{c}; wherein R_{c} is selected from H or

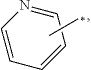

and the asterisk denotes a linkage site;
in formula (I), p is an integer selected from 0 to 5;
in formula (I), the dashed line denotes a single bond or nothing exists; and
a substituent for substitution is selected from any one of C1-C10 alkyl, C3-C10 cycloalkyl, C1-C6 alkoxy, C6-C60 aryl, or C6-C60 heteroaryl.

In a second aspect, the present application provides an OLED display panel, and the OLED display panel comprises a first electrode, a second electrode, and an organic thin film layer provided between the first electrode and the second electrode; the organic thin film layer comprises a light-emitting layer; and a host material of the light-emitting layer comprises any one or a combination of at least two of the organic compounds described in the first aspect.

In a third aspect, the present application provides an electronic device, and the electronic device comprises the OLED display panel described in the second aspect.

### BRIEF DESCRIPTION OF DRAWINGS

FIG. 1 shows a schematic structural diagram of an OLED device provided in an embodiment of the present application.

### REFERENCE LISTS

1 substrate
2 ITO anode
3 first hole transport layer
4 second hole transport layer
5 electron blocking layer
6 light-emitting layer
7 first electron transport layer
8 second electron transport layer
9 cathode
10 capping layer

### DETAILED DESCRIPTION

The technical solutions of the present application are further described below in terms of embodiments. It should be apparent to those skilled in the art that the examples are merely used for a better understanding of the present application and should not be regarded as a specific limitation to the present application.

In a first aspect, the present application provides an organic compound, and the organic compound has a structure represented by Formula (I):

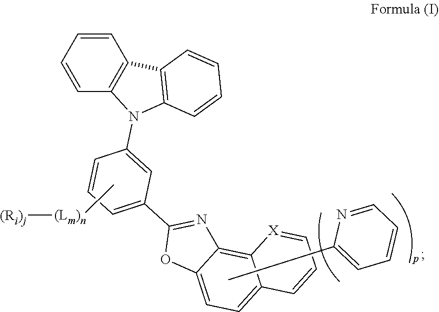

in formula (I), Lₘ is independently selected from any one of a single bond, substituted or unsubstituted C6-C40 (such as C6, C8, C10, C12, C14, C16, C18, C20, C22, C24, C26, C28, C30, C32, C34, C36, C38, C40, etc.) arylene, or substituted or unsubstituted C5-C40 (such as C5, C6, C8, C10, C12, C14, C16, C18, C20, C22, C24, C26, C28, C30, C32, C34, C36, C38, C40, etc.) heteroarylene;
in formula (I), n is an integer selected from 1 to 4 (such as 1, 2, 3, or 4), and m is an integer selected from 1 to n;
in formula (I), Rᵢ is independently selected from any one of substituted or unsubstituted C6-C60 (such as C6, C8, C10, C12, C14, C16, C18, C20, C22, C24, C26, C28, C30, C32, C34, C36, C38, C40, C42, C44, C46, C48, C50, C52, C54, C56, C58, C60 etc.) aryl, substituted or unsubstituted C6-C60 (such as C6, C8, C10, C12, C14, C16, C18, C20, C22, C24, C26, C28, C30, C32, C34, C36, C38, C40, C42, C44, C46, C48, C50, C52, C54, C56, C58, C60, etc.) heteroaryl, substituted or unsubstituted C6-C60 (such as C6, C8, C10, C12, C14, C16, C18, C20, C22, C24, C26, C28, C30, C32, C34, C36, C38, C40, C42, C44, C46, C48, C50, C52, C54, C56, C58, C60, etc.) fused ring aryl, or substituted or unsubstituted C6-C30 (such as C6, C8, C10, C12, C14, C16, C18, C20, C22, C24, C26, C28, C30, etc.) arylamino;
in formula (I), j is an integer selected from 1 to 4 (such as 1, 2, 3, or 4), and the i is an integer selected from 1 to j; and j and n have the same value;
in formula (I), X is selected from N or C—R_{c}; wherein R_{c} is selected from H or

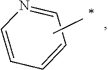

and the asterisk denotes a linkage site;
in formula (I), p is an integer selected from 0 to 5 (such as 0, 1, 2, 3, 4, or 5);
in formula (I), the dashed line denotes a single bond or nothing exists; and
a substituent for substitution is selected from any one of C1-C10 (such as C1, C2, C3, C4, C5, C6, C7, C8, C9, C10) alkyl, C3-C10 (such as C3, C4, C5, C6, C7, C8, C9, C10) cycloalkyl, C1-C6 (such as C1, C2, C3, C4, C5, C6) alkoxy, C6-C60 (such as C6, C8, C10, C12, C14, C16, C18, C20, C22, C24, C26, C28, C30, C32, C34, C36, C38, C40, C42, C44, F46, C48, C50, C52, C54, C56, C58, C60, etc.) aryl, and C6-C60 (such as C6, C8, C10, C12, C14, C16, C18, C20, C22, C24, C26, C28, C30, C32, C34, C36, C38, C40, C42, C44, C46, C48, C50, C52, C54, C56, C58, C60, etc.) heteroaryl. The “substituent for substitution” mentioned in the present application should be interpreted as follows: in a case where a “substituted or unsubstituted” group is a substituted group, a substituent on the substituted group is identified as the “substituent for substitution”; the selection range of species of substituent are described above; the same expression hereinafter has the same meaning. For example, in a case where the Rᵢ is selected from alkyl-substituted aryl, the alkyl group on the aryl group is the “substituent for substitution”.

The organic compound provided by the present application contains an acceptor group and a donor group in its skeleton, which are connected by a benzene ring to form a D-Π-A construction with bipolar transport ability; the benzene ring at the central position of the skeleton can reduce the planarity of the molecule and increase the rigidity; accordingly, the organic compound has a high glass transition temperature and thermal stability, and is easy to form a good amorphous thin film; when used in OLED devices as the host material of the light-emitting layer, the organic compound can reduce the driving voltage of the devices and improve the luminous efficacy and lifetime of the devices.

In one embodiment, Lₘ is independently selected from any one of a single bond, phenylene, biphenylene, naphthylene, anthrylene, or phenanthrylene.

In one embodiment, Lₘ is independently selected from any one of a single bond, phenylene, or biphenylene.

In one embodiment, Lₘ is independently selected from a single bond or any one of the following groups:

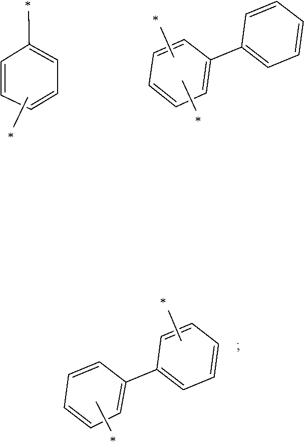

wherein the asterisk denotes a linkage site.

In one embodiment, Lₘ is independently selected from a single bond or any one of the following groups:

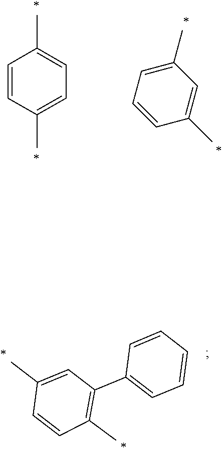

wherein the asterisk denotes a linkage site.

In the present application, Lₘ is preferably selected from the above structures, whereby the planarity of the molecule is reduced and the rigidity of the organic compound is increased.

In one embodiment, n is an integer selected from 1 to 3.

In one embodiment, Rᵢ is independently selected from any one of substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted 9,10-benzophenanthryl, substituted or unsubstituted pyridyl, substituted or unsubstituted quinolyl, substituted or unsubstituted benzoxazolyl, substituted or unsubstituted carbazolyl, or substituted or unsubstituted arylamino; and
a substituent for substitution is selected from any one of C1-C10 alkyl, C3-C10 cycloalkyl, C1-C6 alkoxy, C6-C60 aryl, or C6-C60 heteroaryl.

In one embodiment, Rᵢ is independently selected from any one of substituted or unsubstituted phenyl, substituted or unsubstituted biphenyl, substituted or unsubstituted terphenyl, substituted or unsubstituted naphthyl, substituted or unsubstituted 9,10-benzophenanthryl, substituted or unsubstituted pyridyl, substituted or unsubstituted quinolyl, substituted or unsubstituted benzoxazolyl, substituted or unsubstituted carbazolyl, or substituted or unsubstituted arylamino; and
a substituent for substitution is selected from any one of C1-C10 alkyl, C3-C10 cycloalkyl, C1-C6 alkoxy, C6-C60 aryl, or C6-C60 heteroaryl.

In one embodiment, Rᵢ is independently selected from any one of the following groups:

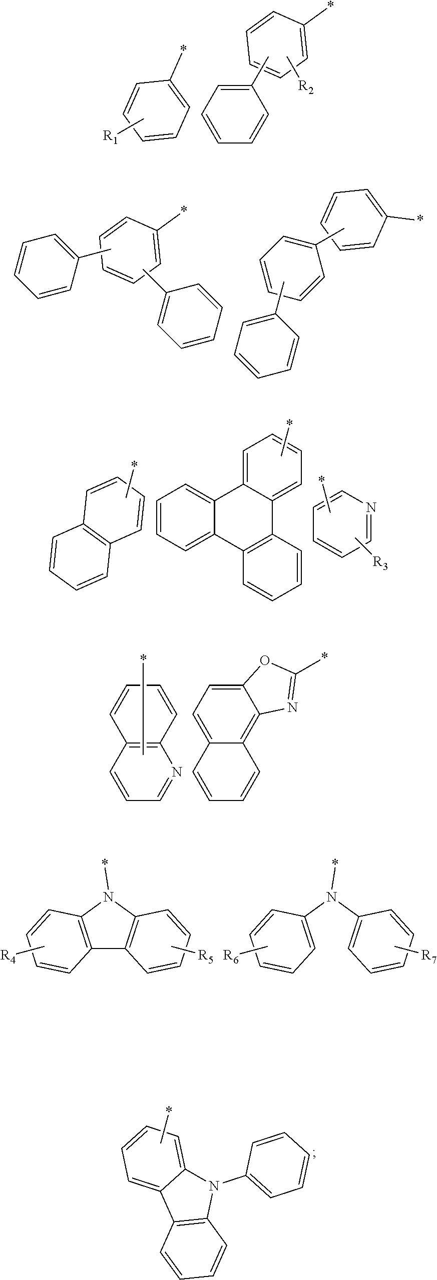

wherein R₁ to R₇ are each independently selected from any one of hydrogen, C1-C10 alkyl, C3-C10 cycloalkyl, C1-C6 alkoxy, C6-C60 aryl, or C6-C60 heteroaryl; and
the asterisk denotes a linkage site.

In the present application, Rᵢ is preferably selected from the above structures, which on the one hand enhances the transmission effect of holes or electrons, facilitating the holes and electrons rapidly recombining in the light-emitting layer, and on the other hand, increases steric hindrance, and reduces the quenching of excitons in the main light-emitting material.

In one embodiment, R₁ to R₇ are each independently selected from any one of hydrogen, C1-C10 alkyl, C3-C10 cycloalkyl, or C6-C60 aryl.

In one embodiment, R₁ and R₂ are each independently selected from any one of hydrogen, methyl, ethyl, propyl, n-butyl, isobutyl, tert-butyl, or cyclohexyl;
R₃ is selected from any one of hydrogen, methyl, ethyl, propyl, or phenyl; and
R₄ to R₇ are each independently selected from any one of hydrogen, methyl, ethyl, propyl, n-butyl, isobutyl, tert-butyl, cyclohexyl, or phenyl.

In one embodiment, Rᵢ is each independently selected from any one of the following groups:

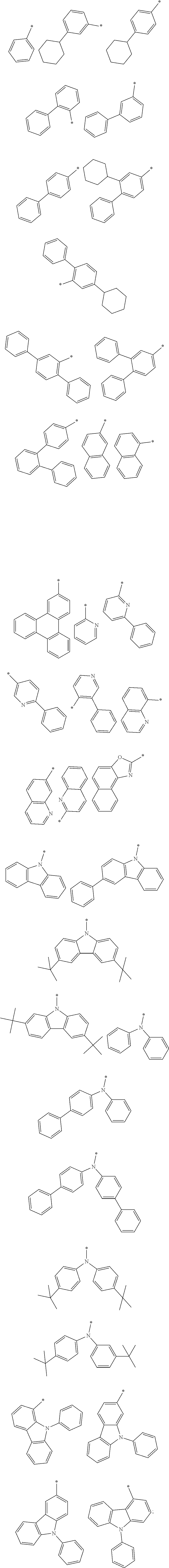

wherein the asterisk denotes a linkage site.

In the present application, Rᵢ is further preferably selected from the above structures, which on the one hand enhances the transmission effect of holes or electrons, facilitating the holes and electrons rapidly recombining in the light-emitting layer, and on the other hand, increases steric hindrance and reduces the quenching of excitons in the main light-emitting material.

In one embodiment, j is an integer selected from 1 to 3.

In one embodiment, R_{c} is selected from H or

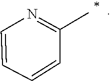

In the present application, R_{c} is preferably pyridyl, increasing the electron transport capacity of the organic compound.

In one embodiment, in a case where X is selected from C—R_{c} and R_{c} is selected from H, p is not 0.

In one embodiment, the organic compound is selected from any one of the following compounds:

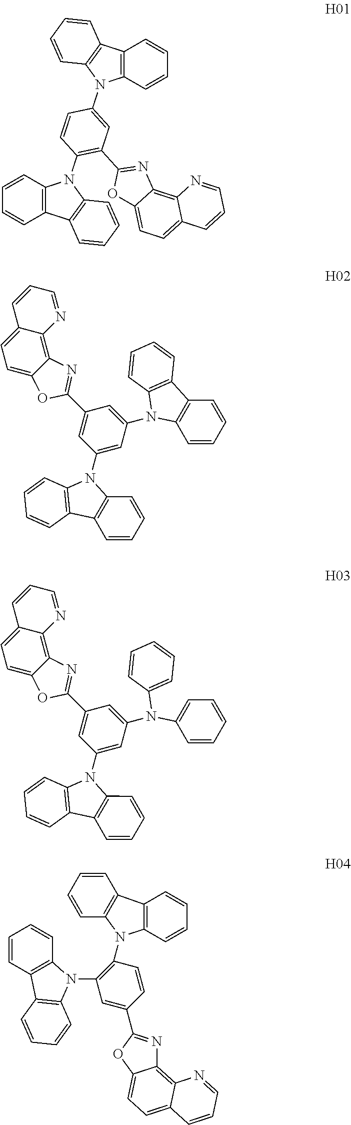

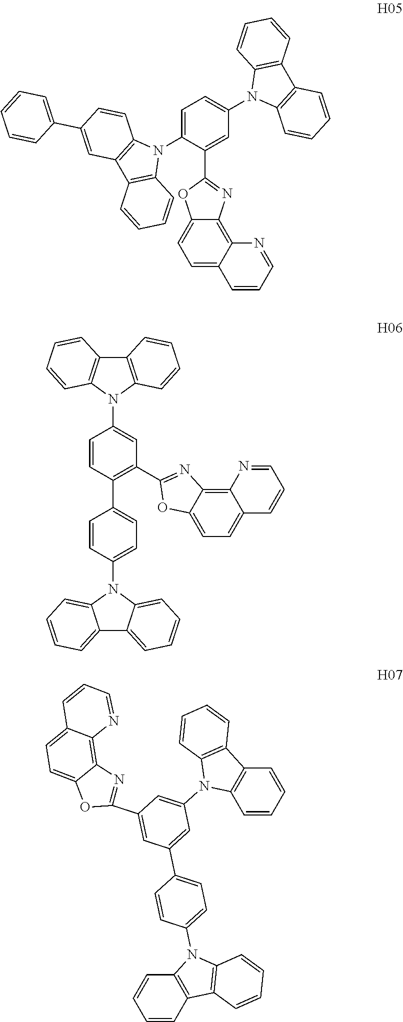

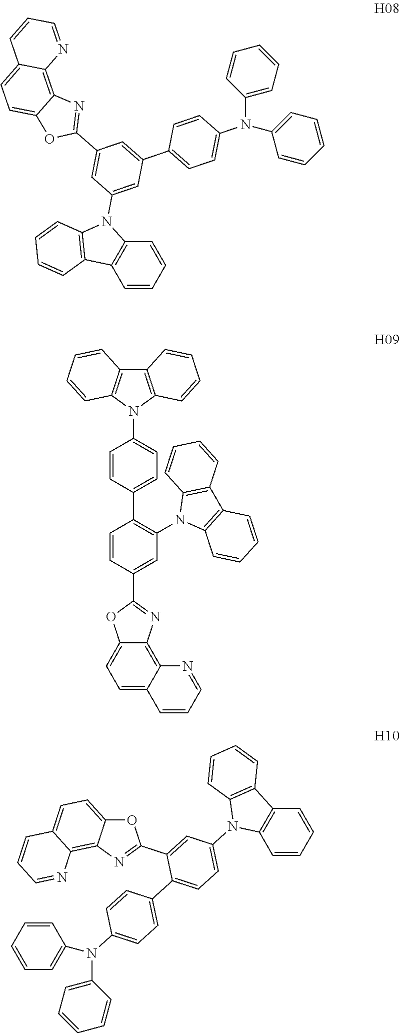 [SMILES: c1ccc(N(c2ccccc2)c2ccc(-c3cc(-c4nc5c(ccc6cccnc65)o4)cc(-n4c5ccccc5c5ccccc54)c3)cc2)cc1 ; c1cnc2c(c1)ccc1oc(-c3ccc(-c4ccc(-n5c6ccccc6c6ccccc65)cc4)c(-n4c5ccccc5c5ccccc54)c3)nc12 ; c1ccc(N(c2ccccc2)c2ccc(-c3ccc(-n4c5ccccc5c5ccccc54)cc3-c3nc4c(ccc5cccnc54)o3)cc2)cc1]

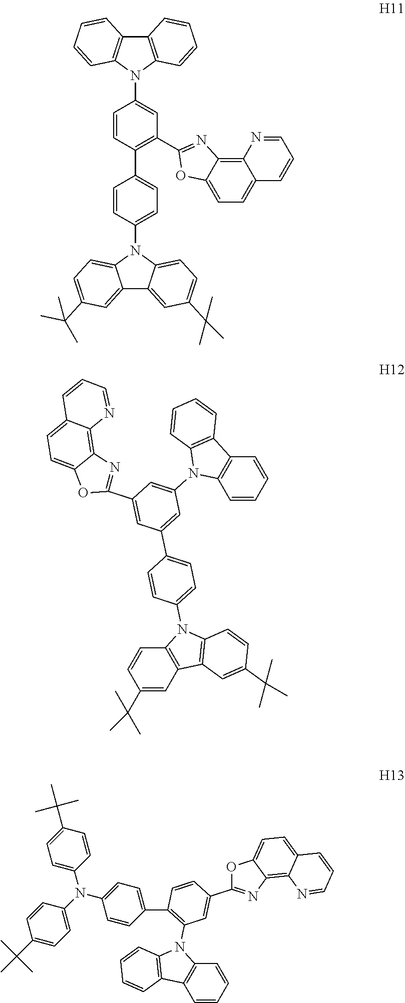

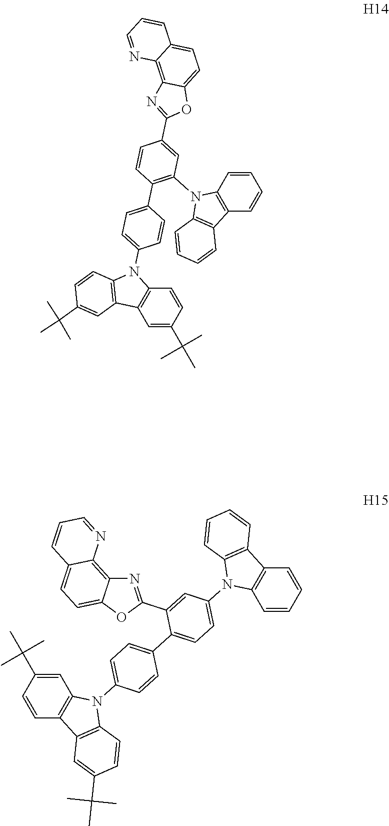 [SMILES: CC(C)(C)c1ccc2c(c1)c1cc(C(C)(C)C)ccc1n2-c1ccc(-c2ccc(-c3nc4c(ccc5cccnc54)o3)cc2-n2c3ccccc3c3ccccc32)cc1 ; CC(C)(C)c1ccc2c(c1)c1ccc(C(C)(C)C)cc1n2-c1ccc(-c2ccc(-n3c4ccccc4c4ccccc43)cc2-c2nc3c(ccc4cccnc43)o2)cc1]

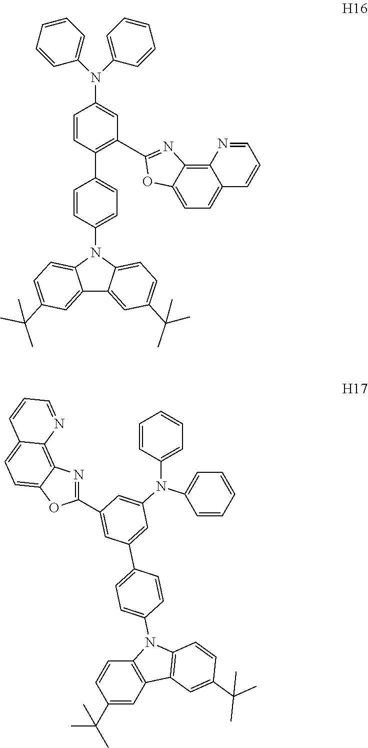 [SMILES: CC(C)(C)c1ccc2c(c1)c1cc(C(C)(C)C)ccc1n2-c1ccc(-c2ccc(N(c3ccccc3)c3ccccc3)cc2-c2nc3c(ccc4cccnc43)o2)cc1 ; CC(C)(C)c1ccc2c(c1)c1cc(C(C)(C)C)ccc1n2-c1ccc(-c2cc(-c3nc4c(ccc5cccnc54)o3)cc(N(c3ccccc3)c3ccccc3)c2)cc1]

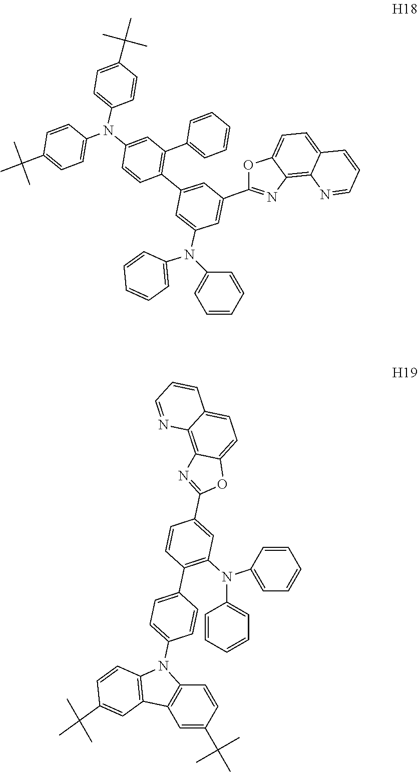

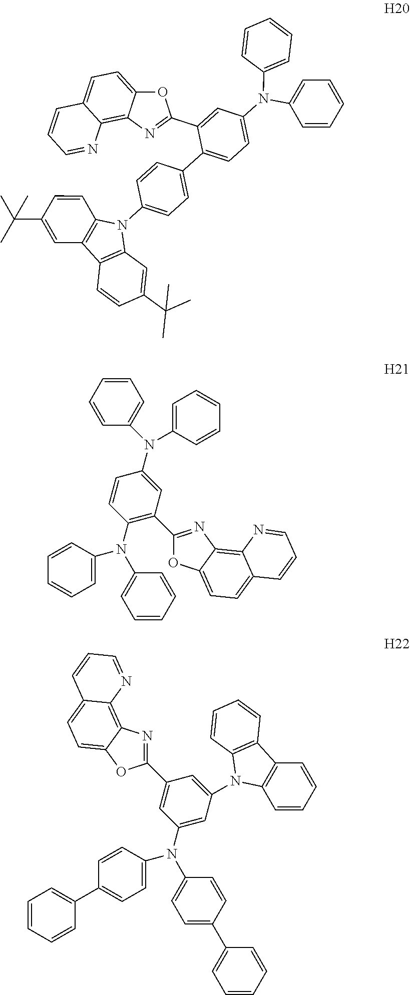 [SMILES: CC(C)(C)c1ccc2c(c1)c1ccc(C(C)(C)C)cc1n2-c1ccc(-c2ccc(N(c3ccccc3)c3ccccc3)cc2-c2nc3c(ccc4cccnc43)o2)cc1 ; c1ccc(N(c2ccccc2)c2ccc(N(c3ccccc3)c3ccccc3)c(-c3nc4c(ccc5cccnc54)o3)c2)cc1 ; c1ccc(-c2ccc(N(c3ccc(-c4ccccc4)cc3)c3cc(-c4nc5c(ccc6cccnc65)o4)cc(-n4c5ccccc5c5ccccc54)c3)cc2)cc1]

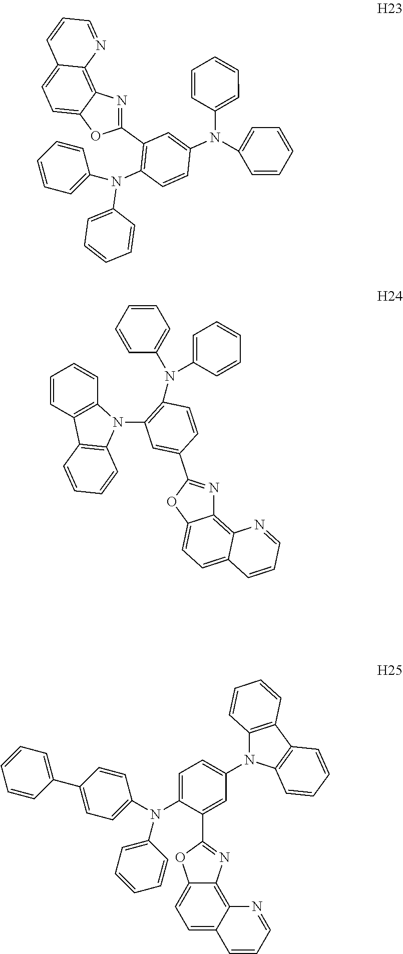 [SMILES: c1ccc(N(c2ccccc2)c2ccc(N(c3ccccc3)c3ccccc3)c(-c3nc4c(ccc5cccnc54)o3)c2)cc1 ; c1ccc(N(c2ccccc2)c2ccc(-c3nc4c(ccc5cccnc54)o3)cc2-n2c3ccccc3c3ccccc32)cc1 ; c1ccc(-c2ccc(N(c3ccccc3)c3ccc(-n4c5ccccc5c5ccccc54)cc3-c3nc4c(ccc5cccnc54)o3)cc2)cc1]

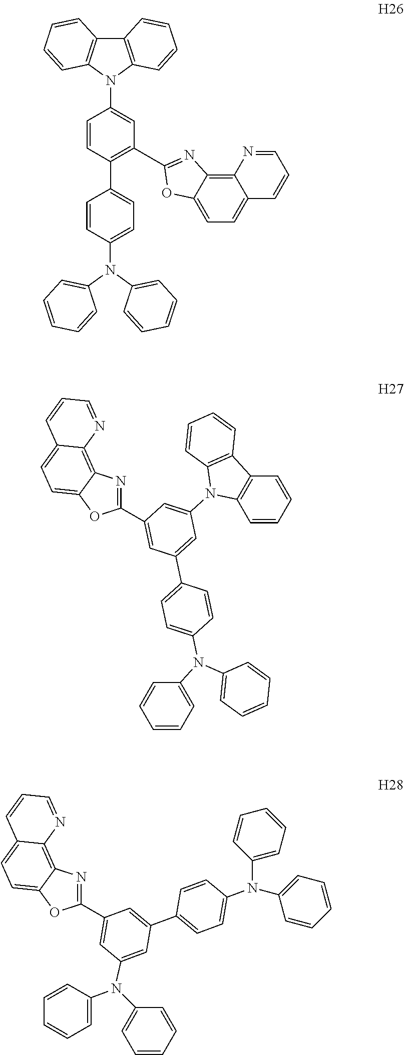 [SMILES: c1ccc(N(c2ccccc2)c2ccc(-c3ccc(-n4c5ccccc5c5ccccc54)cc3-c3nc4c(ccc5cccnc54)o3)cc2)cc1 ; c1ccc(N(c2ccccc2)c2ccc(-c3cc(-c4nc5c(ccc6cccnc65)o4)cc(-n4c5ccccc5c5ccccc54)c3)cc2)cc1 ; c1ccc(N(c2ccccc2)c2ccc(-c3cc(-c4nc5c(ccc6cccnc65)o4)cc(N(c4ccccc4)c4ccccc4)c3)cc2)cc1]

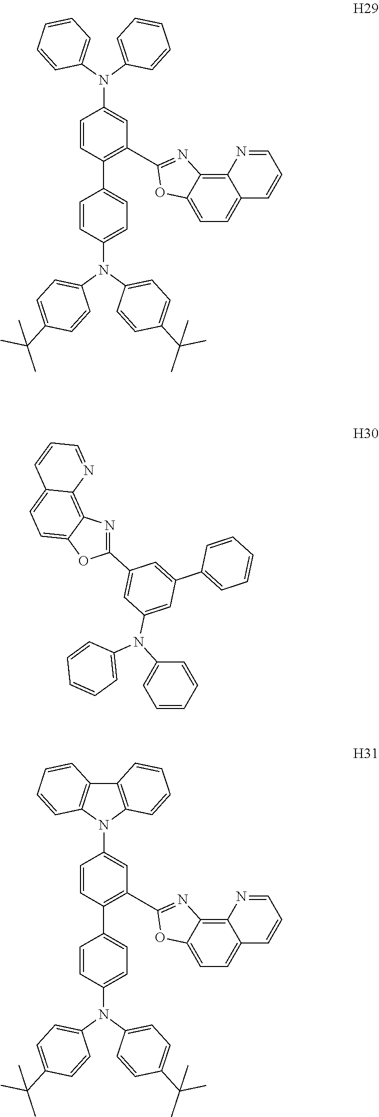 [SMILES: CC(C)(C)c1ccc(N(c2ccc(-c3ccc(N(c4ccccc4)c4ccccc4)cc3-c3nc4c(ccc5cccnc54)o3)cc2)c2ccc(C(C)(C)C)cc2)cc1 ; c1ccc(-c2cc(-c3nc4c(ccc5cccnc54)o3)cc(N(c3ccccc3)c3ccccc3)c2)cc1 ; CC(C)(C)c1ccc(N(c2ccc(-c3ccc(-n4c5ccccc5c5ccccc54)cc3-c3nc4c(ccc5cccnc54)o3)cc2)c2ccc(C(C)(C)C)cc2)cc1]

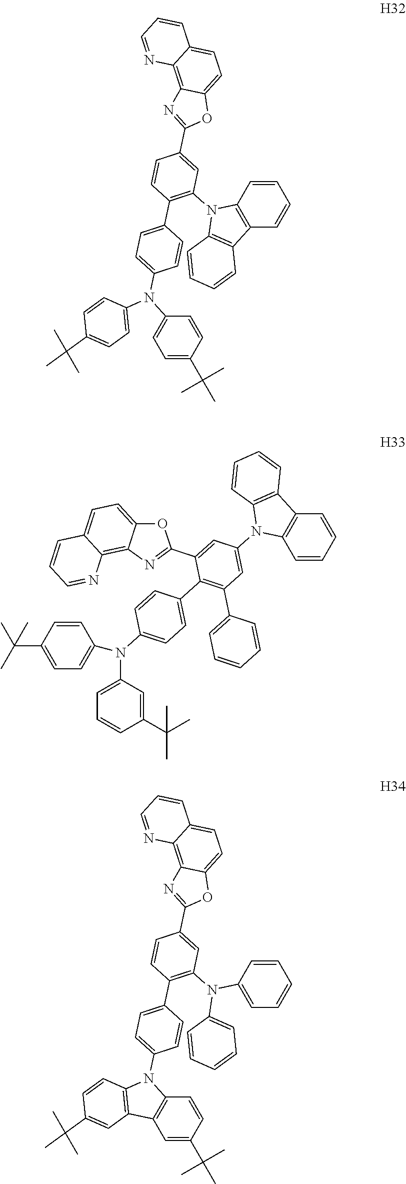

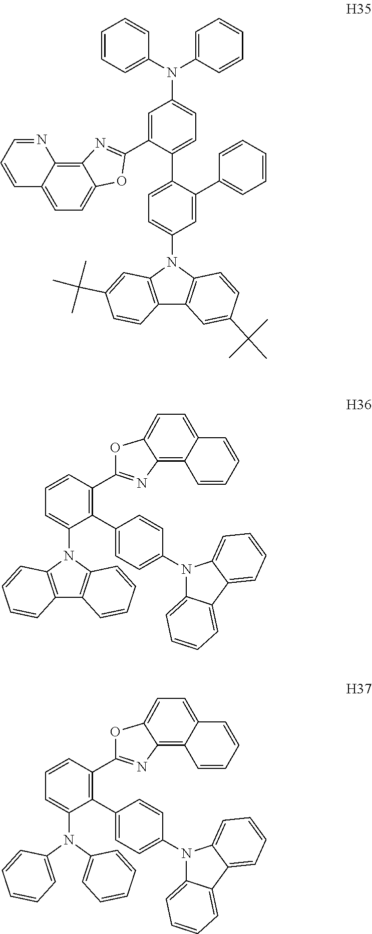 [SMILES: CC(C)(C)c1ccc2c(c1)c1ccc(C(C)(C)C)cc1n2-c1ccc(-c2ccc(N(c3ccccc3)c3ccccc3)cc2-c2nc3c(ccc4cccnc43)o2)c(-c2ccccc2)c1 ; c1cc(-c2nc3c(ccc4ccccc43)o2)c(-c2ccc(-n3c4ccccc4c4ccccc43)cc2)c(-n2c3ccccc3c3ccccc32)c1 ; c1ccc(N(c2ccccc2)c2cccc(-c3nc4c(ccc5ccccc54)o3)c2-c2ccc(-n3c4ccccc4c4ccccc43)cc2)cc1]

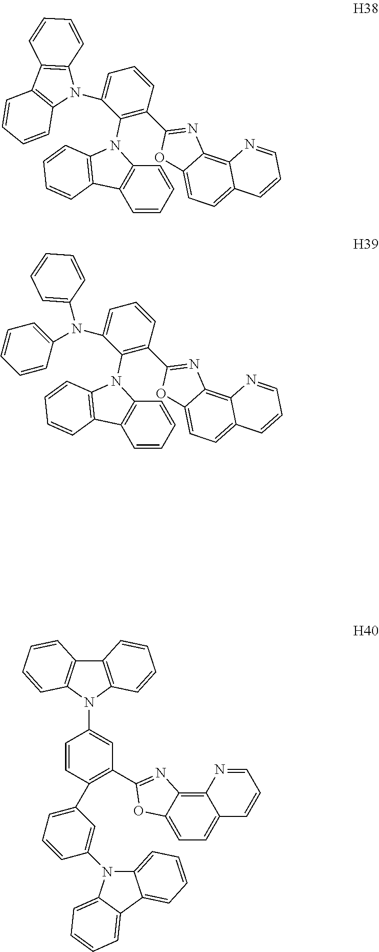 [SMILES: c1cc(-c2nc3c(ccc4cccnc43)o2)c(-n2c3ccccc3c3ccccc32)c(-n2c3ccccc3c3ccccc32)c1 ; c1ccc(N(c2ccccc2)c2cccc(-c3nc4c(ccc5cccnc54)o3)c2-n2c3ccccc3c3ccccc32)cc1 ; c1cc(-c2ccc(-n3c4ccccc4c4ccccc43)cc2-c2nc3c(ccc4cccnc43)o2)cc(-n2c3ccccc3c3ccccc32)c1]

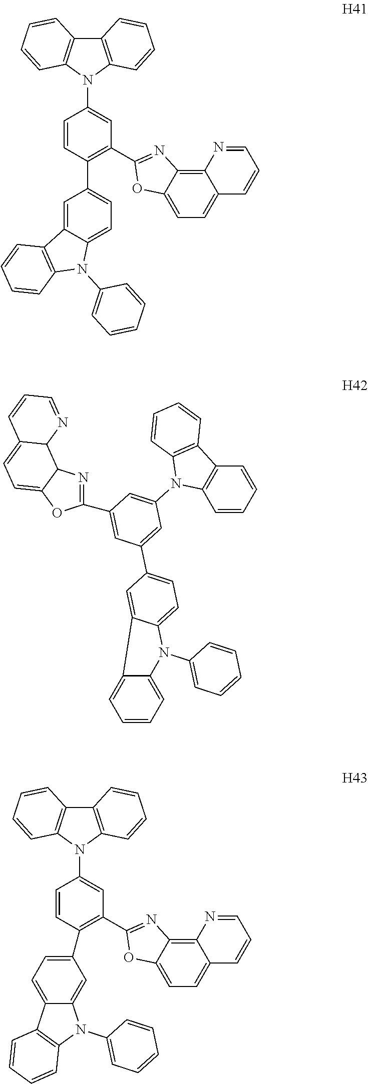 [SMILES: c1ccc(-n2c3ccccc3c3cc(-c4ccc(-n5c6ccccc6c6ccccc65)cc4-c4nc5c(ccc6cccnc65)o4)ccc32)cc1 ; C1=CC2=CC=C3OC(c4cc(-c5ccc6c(c5)c5ccccc5n6-c5ccccc5)cc(-n5c6ccccc6c6ccccc65)c4)=NC3C2N=C1 ; c1ccc(-n2c3ccccc3c3ccc(-c4ccc(-n5c6ccccc6c6ccccc65)cc4-c4nc5c(ccc6cccnc65)o4)cc32)cc1]

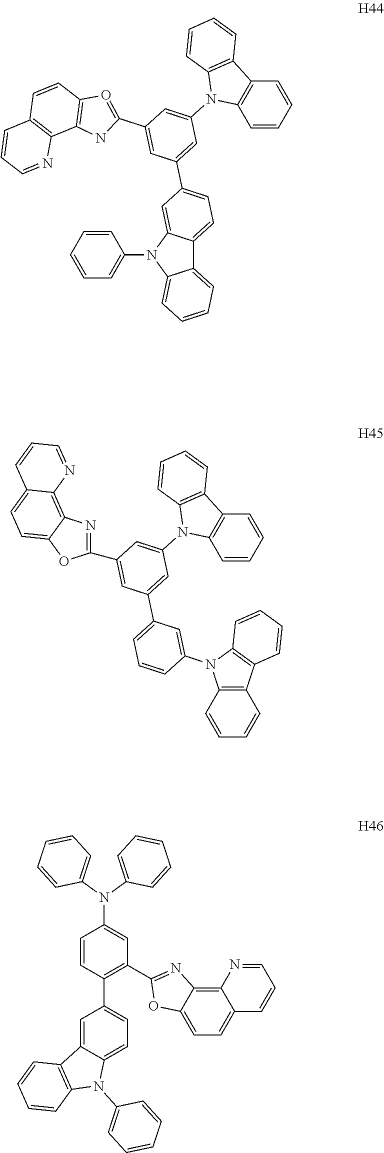

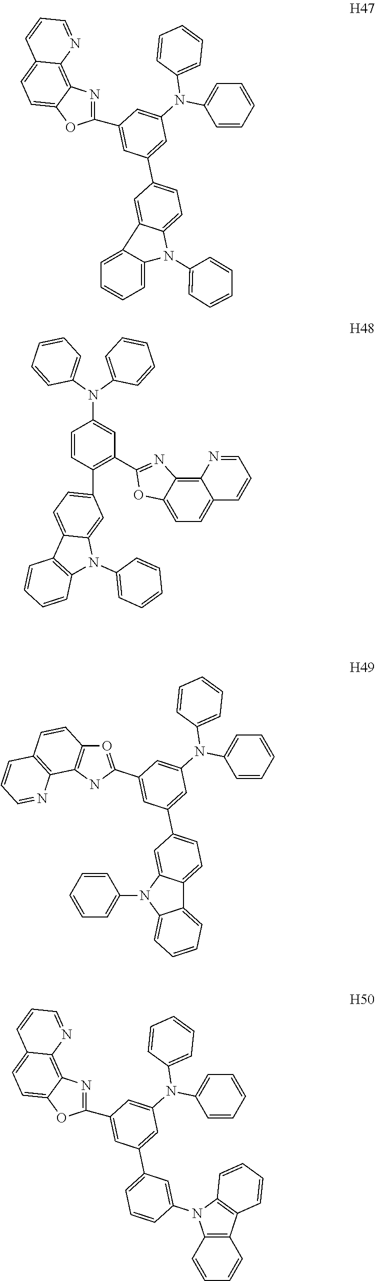

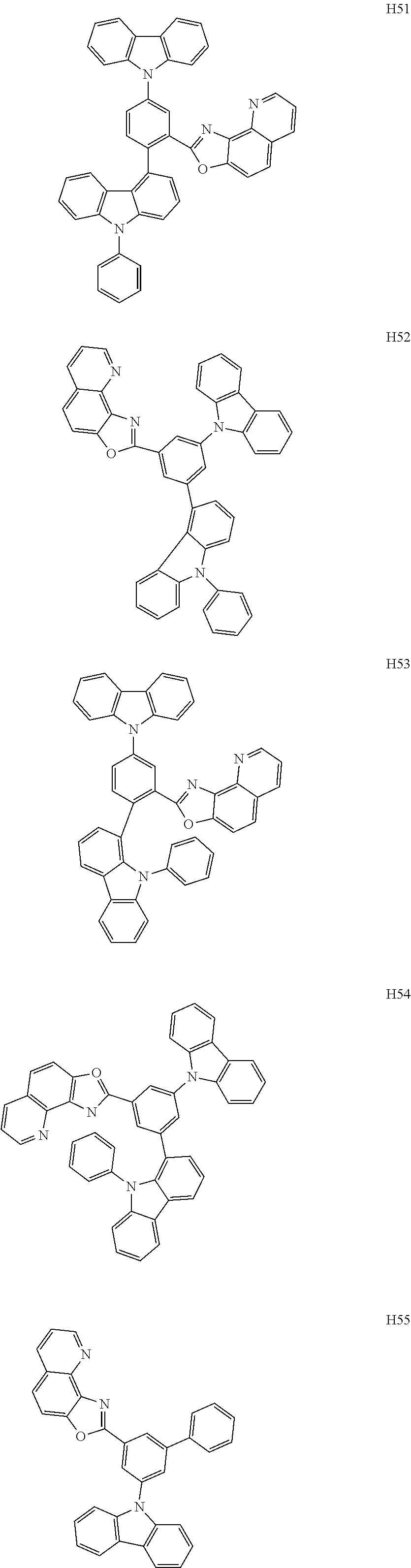

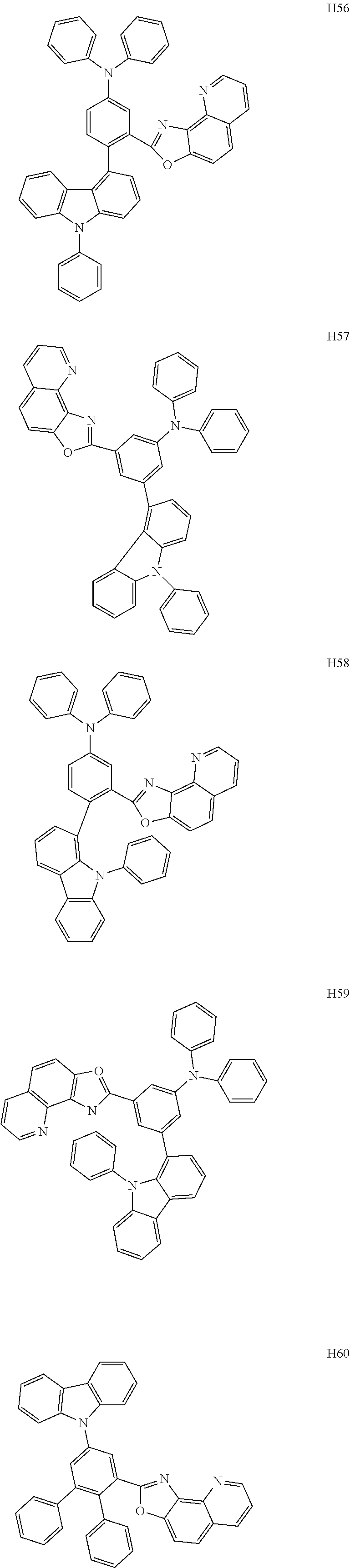

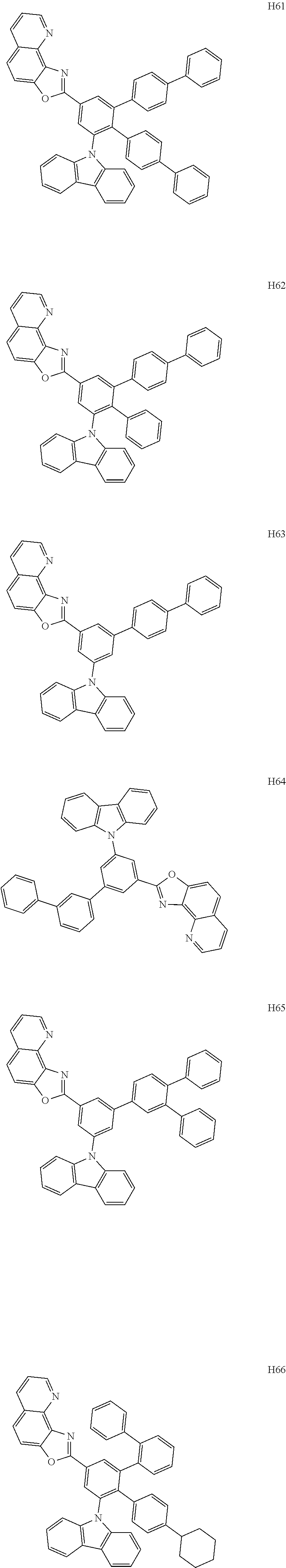

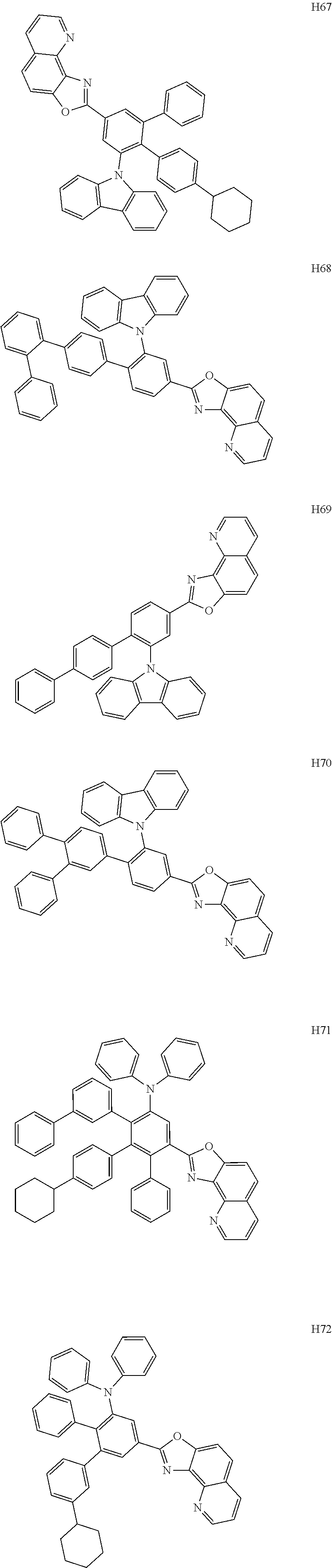

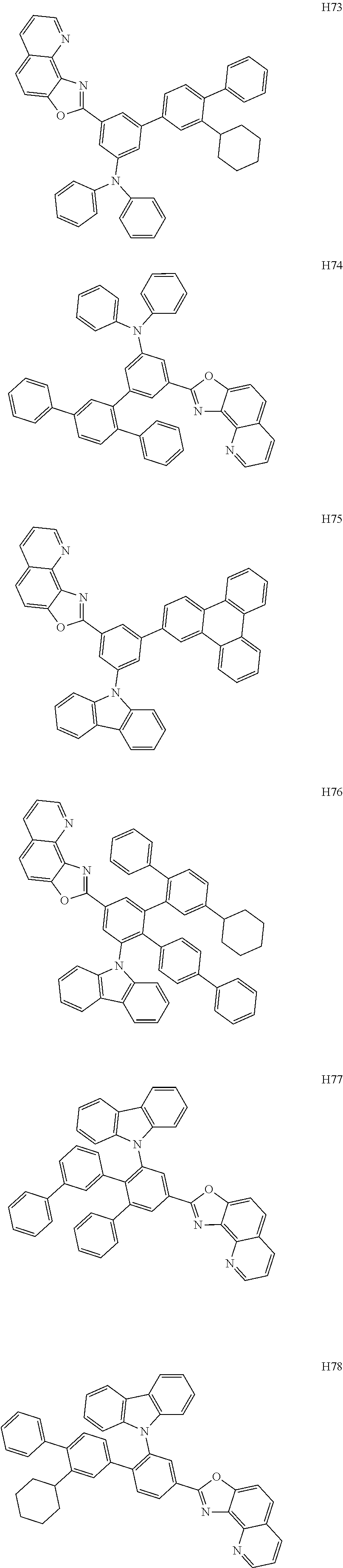

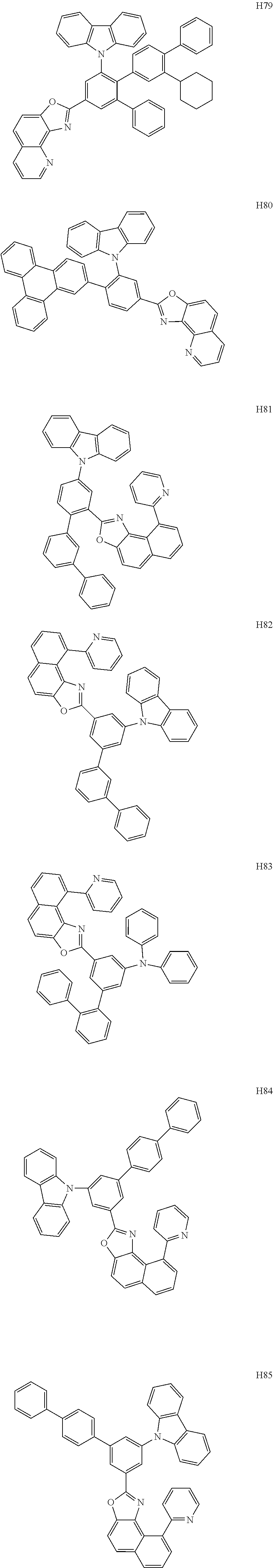

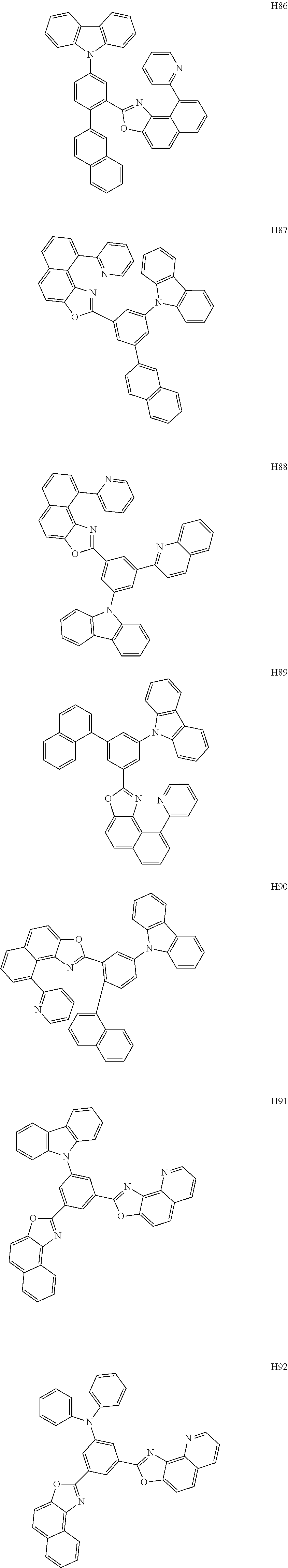

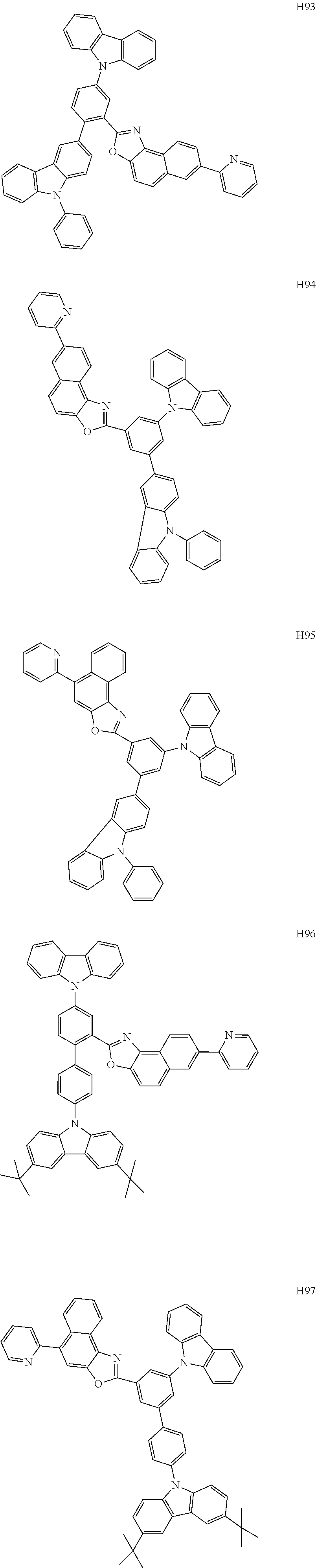

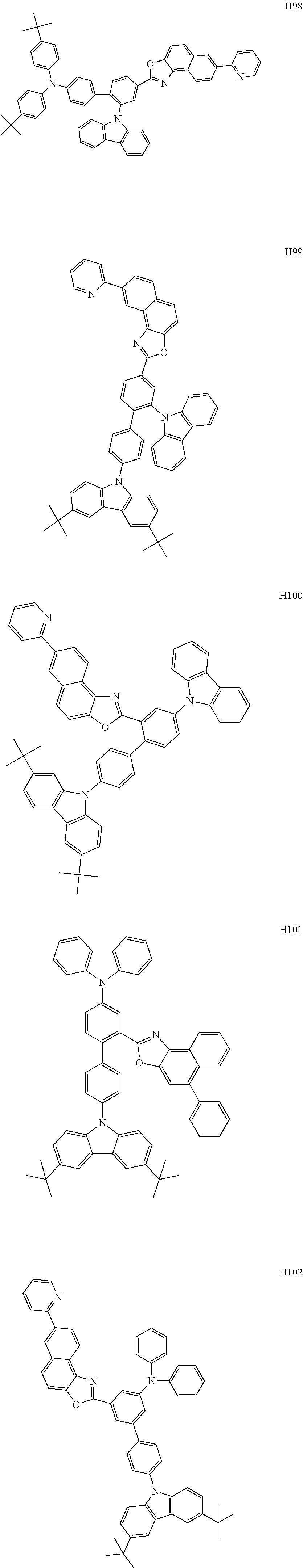

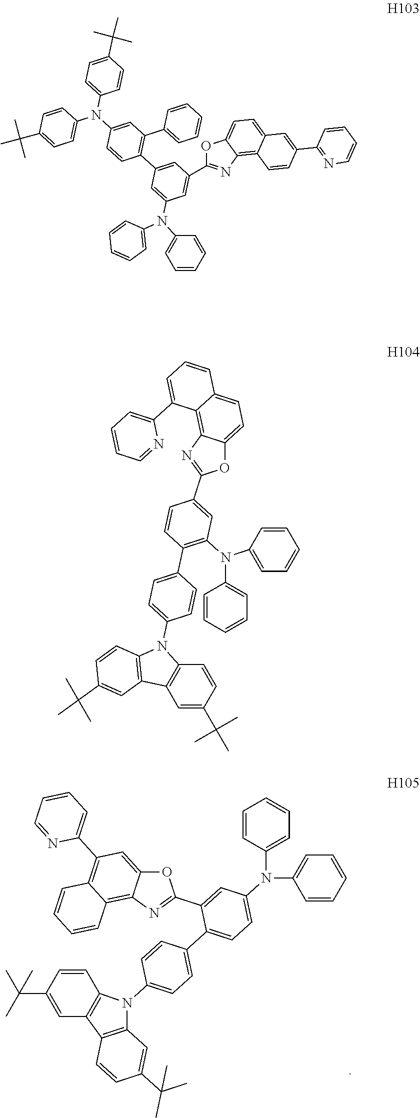 [SMILES: CC(C)(C)c1ccc(N(c2ccc(C(C)(C)C)cc2)c2ccc(-c3cc(-c4nc5c(ccc6cc(-c7ccccn7)ccc65)o4)cc(N(c4ccccc4)c4ccccc4)c3)c(-c3ccccc3)c2)cc1 ; CC(C)(C)c1ccc2c(c1)c1cc(C(C)(C)C)ccc1n2-c1ccc(-c2ccc(-c3nc4c(ccc5cccc(-c6ccccn6)c54)o3)cc2N(c2ccccc2)c2ccccc2)cc1 ; CC(C)(C)c1ccc2c(c1)c1ccc(C(C)(C)C)cc1n2-c1ccc(-c2ccc(N(c3ccccc3)c3ccccc3)cc2-c2nc3c(cc(-c4ccccn4)c4ccccc43)o2)cc1]

In a second aspect, the present application provides an OLED display panel, and the OLED display panel comprises a first electrode, a second electrode, and an organic thin film layer provided between the first electrode and the second electrode; the organic thin film layer comprises a light-emitting layer; and a host material of the light-emitting layer comprises any one or a combination of at least two of the organic compounds described in the first aspect.

In one embodiment, the organic thin film layer further comprises any one or a combination of at least two of a hole injection layer (HIL), a hole transport layer (HTL), an electron blocking layer (EBL), a hole blocking layer (HBL), an electron transport layer (ETL), or an electron injection layer (EIL).

In the OLED display panel provided by the present application, a material of the first electrode (anode) is optionally selected from metal, such as copper, gold, silver, iron, chromium, nickel, manganese, palladium, platinum, and alloys thereof. The first electrode material is also optionally selected from metal oxide, such as indium oxide, zinc oxide, indium tin oxide (ITO), indium zinc oxide (IZO), etc. The first electrode material is further optionally selected from a conductive polymer, such as polyaniline, polypyrrole, poly(3-methylthiophene), etc. In addition, the first electrode material may also be selected from materials and combinations thereof that facilitate hole injection other than the above-listed first electrode materials, including known materials suitable for use in the first electrode.

In the OLED display panel provided by the present application, a material of the second electrode (cathode) is optionally selected from metal, such as aluminum, magnesium, silver, indium, tin, titanium, etc. and alloys thereof. The second electrode material is also optionally selected from a multilayer metal material, such as LiF/Al, LiO₂/Al, BaF₂/Al, etc. In addition to the above-listed second electrode materials, the second electrode material may also be selected from materials and combinations thereof that facilitate electron injection, including known materials suitable for use in the second electrode.

The substrate of the OLED display panel is optionally a rigid substrate, such as borosilicate glass, float soda-lime glass, high refractive index glass, stainless steel, etc., or a flexible substrate, such as a polyimide (PI) plastic substrate, a polyethylene terephthalate (PET) plastic substrate, a polyethylene naphthalate (PEN) plastic substrate, a polyethersulfone resin substrate (PES), a polycarbonate plastic substrate (PC), an ultra-thin flexible glass substrate, a metal foil substrate, etc.

In the OLED display panel provided by the present application, a hole-injection material, a hole-transport material, and an electron-blocking material are each independently and optionally selected from 2,2′-dimethyl-N,N′-di-1-naphthyl-N,N′-diphenyl[1,1′-biphenyl]-4,4′-diamine (α-NPD), 4,4′,4″-tris(carbazol-9-yl)triphenylamine (TCTA), 1,3-dicarbazol-9-ylbenzene (mCP), 4,4′-bis(9-carbazolyl)biphenyl (CBP), 3,3′-bis(N-carbazolyl)-1,1′-biphenyl (mCBP), 2,3,6,7,10,11-hexacyano-1,4,5,8,9,12-hexaazatriphenylene (HATCN), 4,4′-cyclohexyl-bis[N,N-bis(4-methylphenyl)aniline (TAPC), N,N′-diphenyl-N,N′-(1-naphthyl)-1,1′-biphenyl-4,4′-diamine (α-NPB), N,N′-bis(naphth-2-yl)-N,N′-bis(phenyl)biphenyl-4,4′-diamine (NPB), poly(3,4-ethylenedioxythiophene)-polystyrene sulfonate (PEDOT:PSS), polyvinylcarbazole (PVK), 9-phenyl-3,9-bicarbazole (CCP), molybdenum trioxide (MoO₃), etc., but are not limited to the above materials.

In the OLED display panel provided by the present application, a hole-blocking material, an electron-transport material, and an electron-injection material are each independently and optionally selected from 2,8-bis(diphenylphosphoryl)dibenzothiophene (PPT), TSPO1, 1,3,5-tris(1-phenyl-1H-benzimidazol-2-yl)benzene (TPBi), 2,8-bis(diphenylphosphoryl) dibenzofuran (PPF), bis[(2-diphenylphosphoryl)phenyl] ether (DPEPO), lithium fluoride (LiF), 4,6-bis(3,5-di(pyridin-3-yl)phenyl)-2-methylpyrimidine (B3PYMPM), 4,7-diphenyl-1,10-phenanthroline (Bphen), 1,3,5-tris[(3-pyridyl)-3-phenyl]benzene (TmPyBP), tris[2,4,6-trimethyl-3-(3-pyridyl)phenyl]borane (3TPYMB), 1,3-bis(3,5-dipyrid-3-ylphenyl)benzene (B₃PYPB), 1,3-bis[3,5-bis(pyridin-3-yl)phenyl]benzene (BMPYPHB), 2,4,6-tris(biphenyl-3-yl)-1,3,5-triazine (T₂T), diphenylbis[4-(pyridin-3-yl)phenyl]silane (DPPS), cesium carbonate (Cs₂O₃), bis(2-methyl-8-hydroxyquinoline-N1,O8)-(1,1′-biphenyl-4-hydroxy)aluminum (BAlq), 8-hydroxyquinoline-lithium (Liq), tris(8-hydroxyquinoline)aluminum (Alq3), etc., but are not limited to the above materials.

In the OLED display panel provided by the present application, the light-emitting layer optionally contains a host material and a guest material, and the host material is optionally selected from one or more of 2,8-bis(diphenylphosphoryl)dibenzothiophene, 4,4′-bis(9-carbazolyl)biphenyl, 3,3′-bis(N-carbazolyl)-1,1′-biphenyl, 2,8-bis(diphenyl phosphoryl)dibenzofuran, bis(4-(9H-carbazolyl-9-yl)phenyl)diphenylsilane, 9-(4-tert-butylphenyl)-3,6-bis(triphenylmethylsilyl)-9H-carbazole, bis(2-(diphenylphosphino)phenyl)ether, 1,3-bis[3,5-bis(pyridin-3-yl)phenyl]benzene, 4,6-bis(3,5-di(pyridin-3-yl)phenyl)-2-methylpyrimidine, 9-(3-(9H-carbazolyl-9-yl)phenyl)-9H-carbazole-3-carbonitrile, 9-phenyl-9-[4-(triphenylsilyl)phenyl]-9H-fluorene, 1,3,5-tris(1-phenyl-1H-benzimidazol-2-yl)benzene, diphenyl[4-(triphenylsilyl)phenyl]phosphine oxide, 4,4′,4″-tris (carbazol-9-yl)triphenylamine, 2,6-dicarbazole-1,5-pyridine, polyvinylcarbazole, and polyfluorene; or any one or a combination of at least two of the organic compounds provided in the first aspect of the present application; the guest material is optionally selected from one or more of a fluorescent material, a phosphorescent material, or thermally activated delayed fluorescence material and aggregation-induced emission material.

In an embodiment of the present application, the OLED display panel is manufactured by the following process: forming an anode (first electrode) on a transparent or opaque, smooth substrate; forming an organic thin layer on the anode, and forming a cathode (second electrode) on the organic thin layer. The formation of organic thin layer optionally adopts known film forming methods such as vapor deposition, sputtering, spin coating, impregnation, ion plating, etc.

In a third aspect, the present application provides an electronic device, and the electronic device comprises an OLED display panel described in the second aspect.

In one embodiment, the electronic device is optionally a cell phone, a computer, an LCD TV, a smart watch, a smart car, a VR, or AR headset, etc.

The present application provides methods for preparing several exemplary organic compounds of the structure of formula (1). In subsequent embodiments, exemplary descriptions are provided for the synthesis of organic compounds. The exemplary organic compounds synthesized and raw materials used are shown in Table 1.

TABLE 1

| Raw material | Raw material | | Raw material | |
|---|---|---|---|---|
| a-x | b-y | Raw material d-h | f | Compound Hj |
| 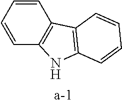 [SMILES: c1ccc2c(c1)[nH]c1ccccc12] | 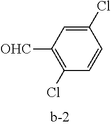 | | 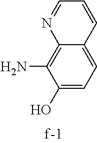 [SMILES: Nc1c(O)ccc2cccnc12] | 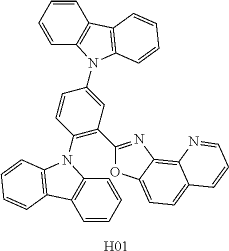 |
| 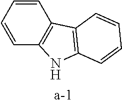 [SMILES: c1ccc2c(c1)[nH]c1ccccc12] | 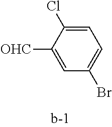 | 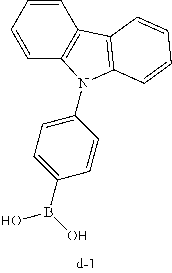 [SMILES: OB(O)c1ccc(-n2c3ccccc3c3ccccc32)cc1] | 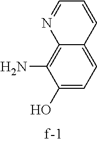 [SMILES: Nc1c(O)ccc2cccnc12] | 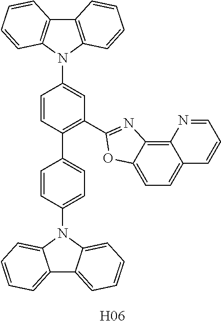 |
| 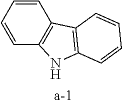 [SMILES: c1ccc2c(c1)[nH]c1ccccc12] | 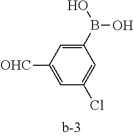 [SMILES: O=Cc1cc(Cl)cc(B(O)O)c1] | 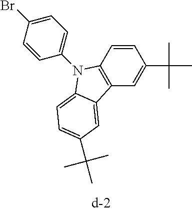 [SMILES: CC(C)(C)c1ccc2c(c1)c1cc(C(C)(C)C)ccc1n2-c1ccc(Br)cc1] | 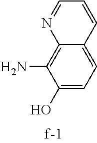 [SMILES: Nc1c(O)ccc2cccnc12] | 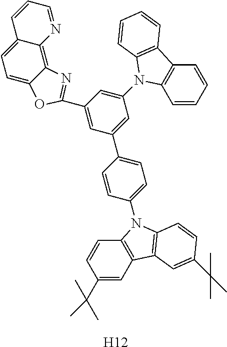 |
| 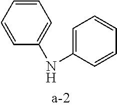 [SMILES: c1ccc(Nc2ccccc2)cc1] | 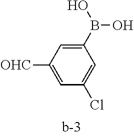 [SMILES: O=Cc1cc(Cl)cc(B(O)O)c1] | 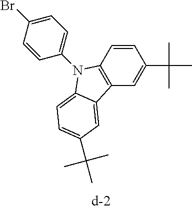 [SMILES: CC(C)(C)c1ccc2c(c1)c1cc(C(C)(C)C)ccc1n2-c1ccc(Br)cc1] | 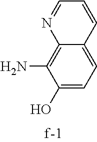 [SMILES: Nc1c(O)ccc2cccnc12] | 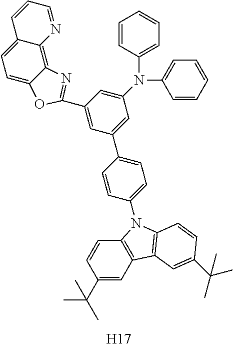 |
| 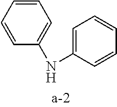 [SMILES: c1ccc(Nc2ccccc2)cc1] | 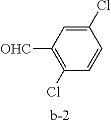 | | 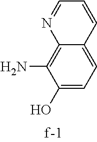 [SMILES: Nc1c(O)ccc2cccnc12] | 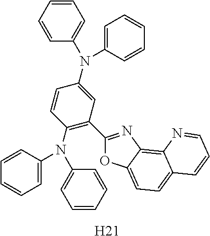 |
| 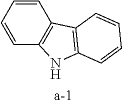 [SMILES: c1ccc2c(c1)[nH]c1ccccc12] | 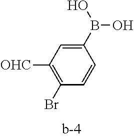 [SMILES: O=Cc1cc(B(O)O)ccc1Br] | 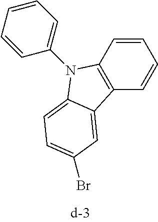 [SMILES: Brc1ccc2c(c1)c1ccccc1n2-c1ccccc1] | 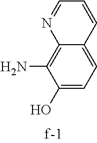 [SMILES: Nc1c(O)ccc2cccnc12] | 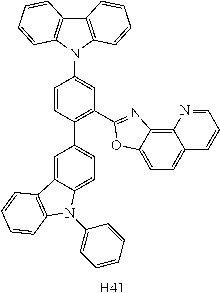 |
| 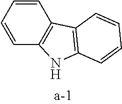 [SMILES: c1ccc2c(c1)[nH]c1ccccc12] | 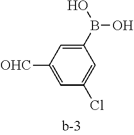 [SMILES: O=Cc1cc(Cl)cc(B(O)O)c1] | 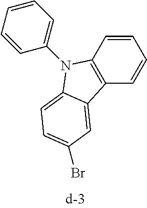 [SMILES: Brc1ccc2c(c1)c1ccccc1n2-c1ccccc1] | 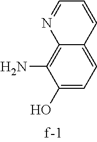 [SMILES: Nc1c(O)ccc2cccnc12] | 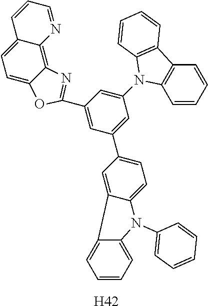 |
| 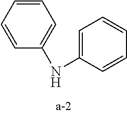 [SMILES: c1ccc(Nc2ccccc2)cc1] | 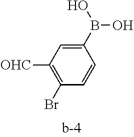 [SMILES: O=Cc1cc(B(O)O)ccc1Br] | 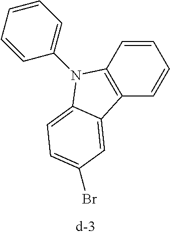 [SMILES: Brc1ccc2c(c1)c1ccccc1n2-c1ccccc1] | 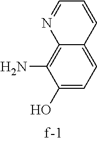 [SMILES: Nc1c(O)ccc2cccnc12] | 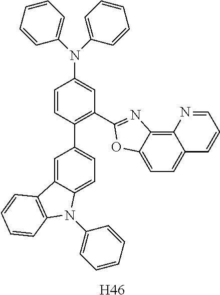 |
| 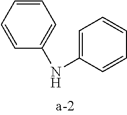 [SMILES: c1ccc(Nc2ccccc2)cc1] | 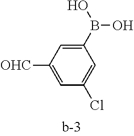 [SMILES: O=Cc1cc(Cl)cc(B(O)O)c1] | 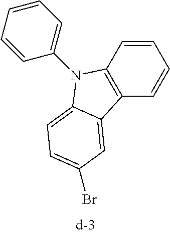 [SMILES: Brc1ccc2c(c1)c1ccccc1n2-c1ccccc1] | 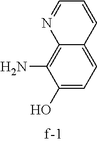 [SMILES: Nc1c(O)ccc2cccnc12] | 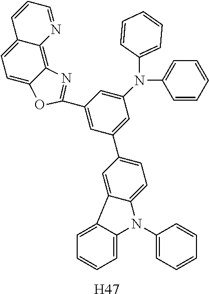 |
| 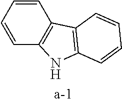 [SMILES: c1ccc2c(c1)[nH]c1ccccc12] | 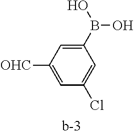 [SMILES: O=Cc1cc(Cl)cc(B(O)O)c1] | 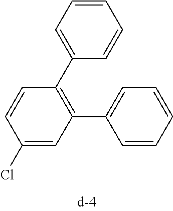 [SMILES: Clc1ccc(-c2ccccc2)c(-c2ccccc2)c1] | 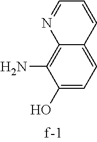 [SMILES: Nc1c(O)ccc2cccnc12] | 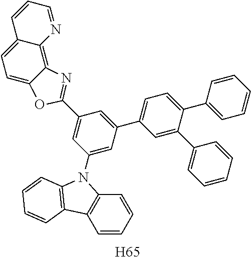 |
| 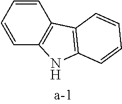 [SMILES: c1ccc2c(c1)[nH]c1ccccc12] | 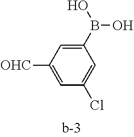 [SMILES: O=Cc1cc(Cl)cc(B(O)O)c1] | 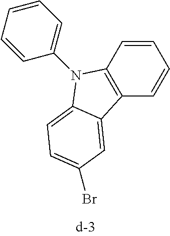 [SMILES: Brc1ccc2c(c1)c1ccccc1n2-c1ccccc1] | 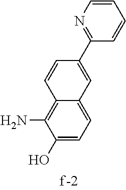 [SMILES: Nc1c(O)ccc2cc(-c3ccccn3)ccc12] | 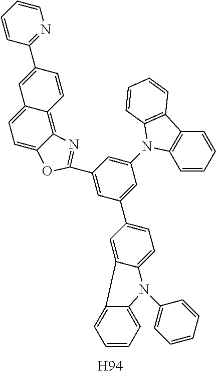 |
| 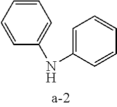 [SMILES: c1ccc(Nc2ccccc2)cc1] | 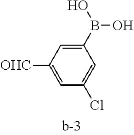 [SMILES: O=Cc1cc(Cl)cc(B(O)O)c1] | 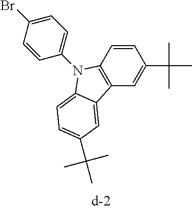 [SMILES: CC(C)(C)c1ccc2c(c1)c1cc(C(C)(C)C)ccc1n2-c1ccc(Br)cc1] | 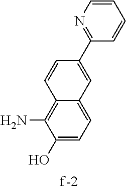 [SMILES: Nc1c(O)ccc2cc(-c3ccccn3)ccc12] | 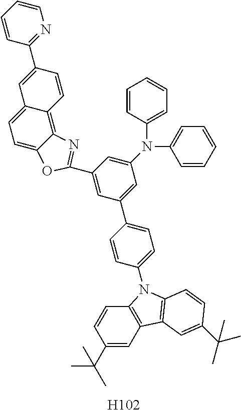 |

### Example 1 Synthesis of Compound H06

### 1) Synthesis of Intermediate c-1

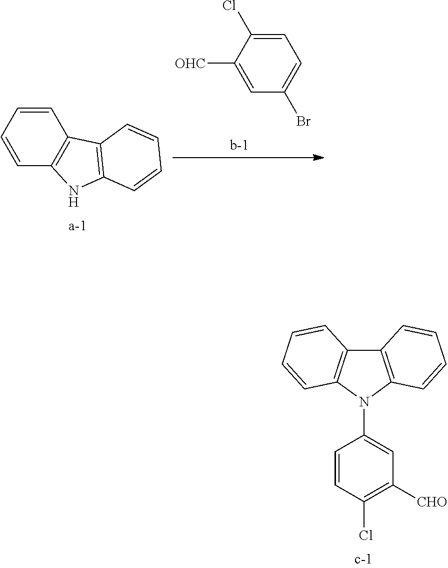 [SMILES: c1ccc2c(c1)[nH]c1ccccc12 ; O=Cc1cc(Br)ccc1Cl ; O=Cc1cc(-n2c3ccccc3c3ccccc32)ccc1Cl]

Under a nitrogen atmosphere, 500 mL of acetonitrile was added to a reaction flask, then Intermediate b-1 (2 mmol), Reactant a-1 (2.1 mmol), K₂CO₃ (7 mmol), catalyst CuI (0.5 mmol), and ligand 18-crown-6 (0.5 mmol) were sequentially added, and the system was heated to 100° C. and reacted for 24 h. After the reaction was completed, the system was cooled to room temperature, and filtered under reduced pressure to collect an organic phase. The organic phase was extracted with 500 mL of dichloromethane/H₂O at 5:5 (v/v) for three times, and the collected organic phase was dried with anhydrous Na₂SO₄, and filtered under reduced pressure to collect a filtrate. The filtrate was subjected to rotary evaporation for solvent removal and then to column chromatography for purification. Intermediate c-1 was obtained.

### 2) Synthesis of Intermediate e-1

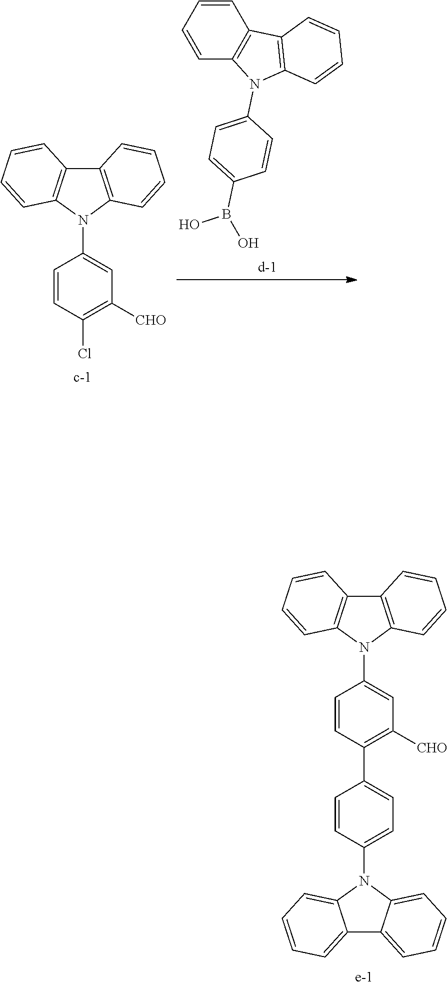 [SMILES: O=Cc1cc(-n2c3ccccc3c3ccccc32)ccc1Cl ; OB(O)c1ccc(-n2c3ccccc3c3ccccc32)cc1 ; O=Cc1cc(-n2c3ccccc3c3ccccc32)ccc1-c1ccc(-n2c3ccccc3c3ccccc32)cc1]

Under a nitrogen atmosphere, Raw material c-1 (0.1 mol), Raw material d-1 (0.12 mol), tetrakis(triphenylphosphine)palladium (0.005 mol), and potassium carbonate (0.3 mol) were mixed with 280 mL of toluene, 60 mL of ethanol, and 60 mL of water, and stirred for 12 hours at 110° C. After the reaction was completed, the mixture was extracted with dichloromethane, and the extract was dried with anhydrous Na₂SO₄, and filtered. The filtrate was distilled under reduced pressure and then purified over silica gel column. Intermediate e-1 was obtained.

### 3) Synthesis of Compound H06

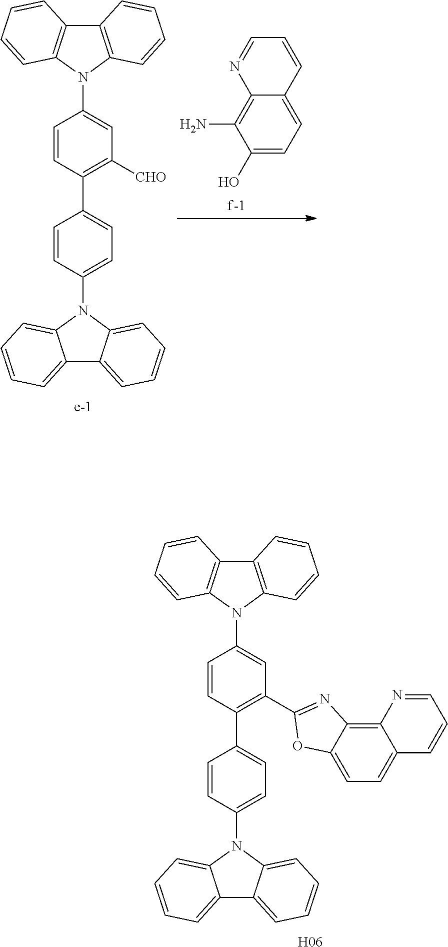

The substances e-1 (40 mmol) and f-1 (60 mmol) were dissolved in glacial acetic acid (700 mL) and reacted for 10 h. After the reaction was completed, the obtained substances were introduced into H₂O (500 mL), and the obtained solid was purified with water and methanol and dried. The obtained compound and 2,3-dichloro-5,6-dicyanobenzoquinone (DDQ) (50 mmol) were dissolved in dichloromethane (300 mL) and reacted for 2 hours after thoroughly dissolution. The reaction mixture was extracted with benzene solvent and a saturated sodium chloride solution. The organic layer was dried with anhydrous magnesium sulfate and then filtered and concentrated. Purification was carried out by column chromatography or distillation to give Compound H06.

### Examples 2-12

Examples 2-12 provide Compound H01, Compound H12, Compound H17, Compound H21, Compound H41, Compound H42, Compound H46, Compound H47, Compound H65, Compound H94, and Compound H102, respectively. See Example 1 for the preparation methods of these compounds, and their respective raw materials and intermediates are shown in Table 1. The difference lies in that the preparation processes of Compounds H01 and H21 employed steps 1) and 3) only, and in step 1) had a molar ratio of a-1:b-2 of 2.1:1 and a molar ratio of a-2:b-2 of 2.1:1, respectively.

The compounds prepared from the above embodiments can be determined by matrix-assisted laser desorption ionization time-of-flight mass spectrometry (abbreviated as MALDI-TOF MS) and elemental analysis, the results of which are shown in Table 2.

TABLE 2

| | | MALDI-TOF | MALDI-TOF | Elemental | Elemental |
|---|---|---|---|---|---|
| | | MS: | MS: | analysis: | analysis: |
| Compound | Molecular | theoretical | measured | theoretical | measured |
| Hj | formula | value (m/z) | value (m/z) | value | value |
| Compound | C₄₀H₂₄ON₄ | 576.20 | 576.64 | C, 83.31; H, 4.20; | C, 83.27; H, 4.22; |
| H01 | | | | O, 2.77; N, 9.72 | O, 2.77; N, 9.74 |
| Compound | C₄₆H₂₈ON₄ | 652.23 | 653.20 | C, 84.64; N, 8.58; | C, 84.60; N, 8.60; |
| H06 | | | | O, 2.45; H, 4.32 | O, 2.47; H, 4.32 |
| Compound | C₅₅H₄₈ON₄ | 780.38 | 781.37 | C, 84.58; N, 7.17; | C, 84.55; N, 7.19; |
| H12 | | | | O, 2.05; H, 6.20 | O, 2.06; H, 6.20 |
| Compound | C₅₅H₅₀ON₄ | 782.40 | 783.43 | C, 84.37; N, 7.16 | C, 84.41; N, 7.14 |
| H17 | | | | O, 2.04; H, 6.44 | O, 2.02; H, 6.44 |
| Compound | C₄₀H₂₈ON₄ | 580.23 | 581.20 | C, 82.74; N, 9.65 | C, 82.76; N, 9.64 |
| H21 | | | | O, 2.76; H, 4.86 | O, 2.75; H, 4.85 |
| Compound | C₄₆H₂₈ON₄ | 652.23 | 653.21 | C, 84.64; N, 8.58 | C, 84.66; N, 8.60 |
| H41 | | | | O, 2.45; H, 4.32 | O, 2.40; H, 4.32 |
| Compound | C₄₇H₃₂ON₄ | 668.26 | 669.22 | C, 84.41; N, 8.38 | C, 84.40; N, 8.37 |
| H42 | | | | O, 2.39; H, 4.82 | O, 2.37; H, 4.86 |
| Compound | C₄₆H₃₀ON₄ | 654.24 | 655.23 | C, 84.38; N, 8.56 | C, 84.39; N, 8.57 |
| H46 | | | | O, 2.44; H, 4.62 | O, 2.42; H, 4.61 |
| Compound | C₄₇H₃₄ON₄ | 670.27 | 671.26 | C, 84.15; N, 8.35 | C, 84.10; N, 8.34 |
| H47 | | | | O, 2.39; H, 5.11 | O, 2.40; H, 5.16 |
| Compound | C₄₆H₂₉ON₃ | 639.23 | 640.14 | C, 86.36; N, 6.57 | C, 86.34; N, 6.55 |
| H65 | | | | O, 2.50; H, 4.57 | O, 2.52; H, 4.59 |
| Compound | C₅₃H₃₆ON₄ | 744.29 | 744.20 | C, 85.46; H, 4.87 | C, 85.44; H, 4.88 |
| H94 | | | | O, 2.15; N, 7.52 | O, 2.16; N, 7.52 |
| Compound | C₆₁H₅₄ON₄ | 858.43 | 858.50 | C, 85.28; H, 6.34 | C, 85.25; H, 6.37 |
| H102 | | | | O, 1.86; N, 6.52 | O, 1.85; N, 6.53 |

### Application Example 1

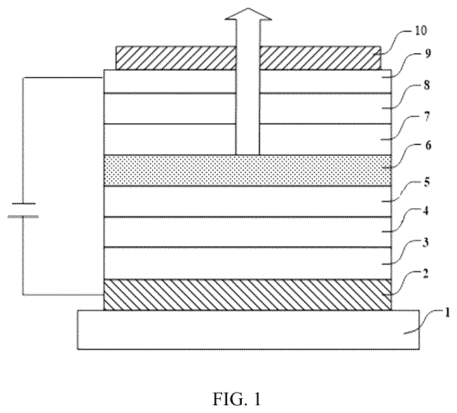

This application example provides an OLED device; its schematic structural diagram is shown in FIG. 1. The organic electroluminescent device comprises: a substrate 1, an ITO anode 2, a first hole transport layer 3, a second hole transport layer 4, an electron blocking layer 5, a light-emitting layer 6, a first electron transporting layer 7, a second electron transporting layer 8, a cathode 9 (a magnesium-silver electrode with a magnesium-silver mass ratio of 9:1), and a capping layer (CPL) 10, wherein the ITO anode 2 has a thickness of 15 nm, the first hole transport layer 3 has a thickness of 10 nm, the second hole transport layer 4 has a thickness of 95 nm, the electron blocking layer 5 has a thickness of 30 nm, the light-emitting layer 6 has a thickness of 30 nm, the first electron transport layer 7 has a thickness of 30 nm, the second electron transport layer 8 has a thickness of 5 nm, the magnesium-silver electrode 9 has a thickness of 15 nm, and the capping layer (CPL) 10 has a thickness of 100 nm. The arrow in FIG. 1 denotes the direction of light emission from the organic electroluminescent device.

The OLED device is prepared according to the following steps:
(1) the glass substrate 1 was cut into a size of 50 mm×50 mm×0.7 mm, processed by ultrasound for 30 min in isopropanol and deionized water, respectively, and then exposed to ozone for about 10 min for cleaning; the resulting glass substrate with ITO anode 2 was loaded onto vacuum deposition equipment;
(2) on the ITO anode 2, HAT-CN and HT-1, hole buffer layer materials, were deposited with thicknesses of 2 nm and 8 nm, respectively, via vacuum evaporation, and this layer served as the first hole transport layer 3;
(3) the material HT-1 for the second hole transport layer 4 was deposited by vacuum evaporation on the first hole transport layer 3 to obtain a layer of 95 nm thickness, which served as the second hole transport layer 4;
(4) the material Prime-1 was evaporated on the second hole transport layer 4 to obtain a layer of 30 nm thickness, which served as the electron blocking layer 5;
(5) the light-emitting layer 6 was formed on the electron blocking layer 5 via co-deposition with a thickness of 30 nm, wherein the organic compound H01 provided by the present application was used as the host material and Ir(piq)₂(acac) was used as the dopant material, and a mass ratio of the organic compound H01 to Ir(piq)₂(acac) was 19:1;
(6) the compound ET-1 for the first electron transport layer 7 was deposited by vacuum evaporation on the light-emitting layer 6 to obtain the first electron transport layer 7 having a thickness of 30 nm;
(7) the material LiF for the second electron transport layer 8 was deposited by vacuum evaporation on the first electron transport layer 7 to obtain the second electron transport layer 8 having a thickness of 5 nm;
(8) magnesium and silver were deposited by vacuum evaporation on the second electron transport layer 8 to obtain the cathode 9 having a thickness of 15 nm, wherein a mass ratio of Mg:Ag is 9:1; and
(9) the hole-type material CPL-1 with a high refractive index was deposited by vacuum evaporation by a thickness of 100 nm on the cathode 9 to serve as the cathode cover layer (capping layer or CPL) 10.

The structural formulas of the materials HAT-CN, HT-1, Prime-1, Ir(piq)₂(acac), ET-1, and CPL-1 mentioned in the above steps are shown as follows:

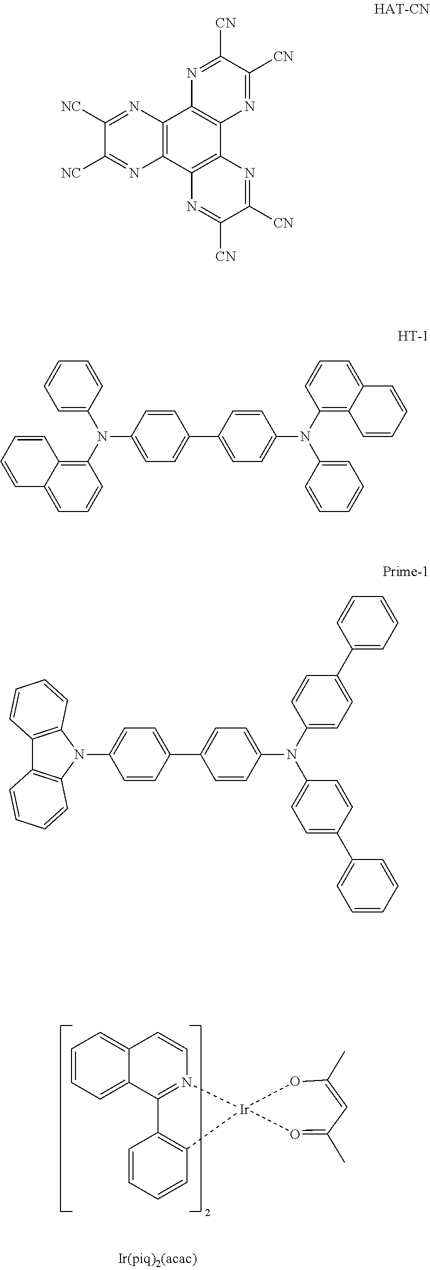

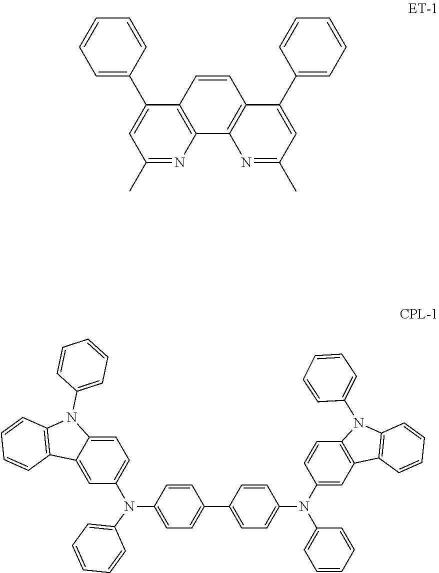 [SMILES: Cc1cc(-c2ccccc2)c2ccc3c(-c4ccccc4)cc(C)nc3c2n1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

### Application Example 2

This application example differs from Application Example 1 only in that the organic compound H01 in step (5) was replaced with an equivalent mass of the organic compound H06 provided by the present application; all other preparation steps are the same.

### Application Example 3

This application example differs from Application Example 1 only in that the organic compound H01 in step (5) was replaced with an equivalent mass of the organic compound H12 provided by the present application; all other preparation steps are the same.

### Application Example 4

This application example differs from Application Example 1 only in that the organic compound H01 in step (5) was replaced with an equivalent mass of the organic compound H17 provided by the present application; all other preparation steps are the same.

### Application Example 5

This application example differs from Application Example 1 only in that the organic compound H01 in step (5) was replaced with an equivalent mass of the organic compound H21 provided by the present application; all other preparation steps are the same.

### Application Example 6

This application example differs from Application Example 1 only in that the organic compound H01 in step (5) was replaced with an equivalent mass of the organic compound H41 provided by the present application; all other preparation steps are the same.

### Application Example 7

This application example differs from Application Example 1 only in that the organic compound H01 in step (5) was replaced with an equivalent mass of the organic compound H42 provided by the present application; all other preparation steps are the same.

### Application Example 8

This application example differs from Application Example 1 only in that the organic compound H01 in step (5) was replaced with an equivalent mass of the organic compound H46 provided by the present application; all other preparation steps are the same.

### Application Example 9

This application example differs from Application Example 1 only in that the organic compound H01 in step (5) was replaced with an equivalent mass of the organic compound H47 provided by the present application; all other preparation steps are the same.

### Application Example 10

This application example differs from Application Example 1 only in that the organic compound H01 in step (5) was replaced with an equivalent mass of the organic compound H65 provided by the present application; all other preparation steps are the same.

### Application Example 11

This application example differs from Application Example 1 only in that the organic compound H01 in step (5) was replaced with an equivalent mass of the organic compound H94 provided by the present application; all other preparation steps are the same.

### Application Example 12

This application example differs from Application Example 1 only in that the organic compound H01 in step (5) was replaced with an equivalent mass of the organic compound H102 provided by the present application; all other preparation steps are the same.

### Comparative Application Example 1

This comparative application example differs from Application Example 1 only in that the organic compound H01 in step (5) was replaced with an equivalent mass of the comparative compound M1

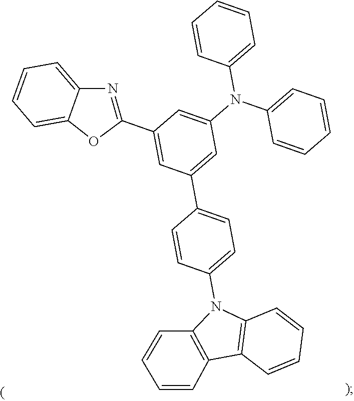 [SMILES: c1ccc(N(c2ccccc2)c2cc(-c3ccc(-n4c5ccccc5c5ccccc54)cc3)cc(-c3nc4ccccc4o3)c2)cc1]

all other preparation steps are the same.

Performance evaluation of OLED devices

A Keithley 2365A digital nanovoltmeter was used for testing currents of the OLED device at different voltages, and then the currents were divided by a luminescence area to obtain current densities of the OLED device at different voltages. A Konicaminolta CS-2000 spectroradiometer was used for testing the brightness and radiation energy flux densities of the OLED device at different voltages. According to the current densities and brightness of the OLED device at different voltages, a working voltage and current efficiency (CE, cd/A) at the same current density (10 mA/cm²) were obtained, where Von denotes the turn-on voltage when the brightness is 1 cd/m². A lifetime LT95 was obtained (under a testing condition of 50 mA/cm²) by measuring the time taken for the OLED device to reach 95% of its initial brightness.

The detailed data are shown in Table 3.

TABLE 3

| | Host material of | | | |
|---|---|---|---|---|
| OLED device | light-emitting layer | Vₒₙ | CE | LT95 |
| Application | H01 | 98.3% | 107.1% | 106.9% |
| Example 1 | | | | |
| Application | H06 | 98.2% | 106.4% | 107.2% |
| Example 2 | | | | |
| Application | H12 | 98.6% | 107.8% | 107.5% |
| Example 3 | | | | |
| Application | H17 | 97.9% | 108.2% | 108.3% |
| Example 4 | | | | |
| Application | H21 | 98.5% | 106.9% | 107.3% |
| Example 5 | | | | |
| Application | H41 | 97.8% | 105.8% | 106.8% |
| Example 6 | | | | |
| Application | H42 | 97.7% | 107.3% | 108.1% |
| Example 7 | | | | |
| Application | H46 | 98.1% | 107.2% | 107.2% |
| Example 8 | | | | |
| Application | H47 | 97.9% | 106.2% | 108.0% |
| Example 9 | | | | |
| Application | H65 | 98.5% | 105.9% | 108.5% |
| Example 10 | | | | |
| Application | H94 | 98.9% | 104.7% | 106.4% |
| Example 11 | | | | |
| Application | H102 | 99.0% | 105.2% | 105.9% |
| Example 12 | | | | |
| Comparative | Comparative | 100.00% | 100.00% | 100.00% |
| Application | Compound M1 | | | |
| Example 1 | | | | |

As can be seen from the data in Table 3, the electroluminescent devices using the organic compounds of the present application have a lower turn-on voltage, which decreases by about 1% to 2.3% (as shown in Table 3, the turn-on voltage is a relative turn-on voltage calculated by setting the turn-on voltage of the device in Comparative Application Example 1 being 100%), and thereby the power consumption of devices can be effectively reduced; the devices using the organic compounds of the present application have a higher current efficiency, which increases by about 4.7% to 8.2% compared with Comparative Application Example 1 (as shown in Table 3, the current efficiency is a relative current efficiency calculated by setting the current efficiency of the device in Comparative Application Example 1 being 100%); the devices using the organic compounds of the present application have a longer lifetime, which is extended by about 5.9% to 8.5% compared with Comparative Application Example 1 (as shown in Table 3, the LT95 is a relative LT95 calculated by setting the LT95 of the device in Comparative Application Example 1 being 100%).

The applicant has stated that although the organic compound and the use thereof in the present application are described through the embodiments, the present application is not limited to the embodiments, which means that the implementation of the present application does not necessarily depend on the embodiments described above. It should be apparent to those skilled in the art that any improvements made to the present application, equivalent replacements of raw materials selected in the present application and addition of adjuvant ingredients thereof, selections of specific methods, etc., shall all fall within the protection scope and disclosed scope of the present disclosure.

What is claimed is:

1. An organic compound, having a structure represented by Formula (I): 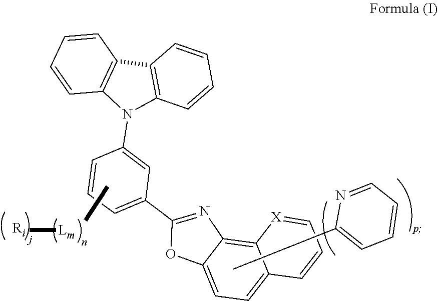
wherein Lₘ is independently selected from any one of a single bond, substituted or unsubstituted C6-C40 arylene, or substituted or unsubstituted C5-C40 heteroarylene;
n is an integer selected from 1 to 4, and m is an integer selected from 1 to n;
Rᵢ is independently selected from any one of substituted or unsubstituted C6-C60 aryl, substituted or unsubstituted C6-C60 heteroaryl, substituted or unsubstituted C6-C60 fused ring aryl, or substituted or unsubstituted C6-C30 arylamino;
j is an integer selected from 1 to 4, and i is an integer selected from 1 to j; and j and n have the same value;
X is selected from N or C—R_{c}; wherein R_{c} is selected from H or 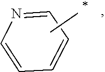
and the asterisk denotes a linkage site;
p is an integer selected from 0 to 5;
in formula (I), the dashed line denotes a single bond or nothing exists; and
a substituent for substitution is selected from any one of C1-C10 alkyl, C3-C10 cycloalkyl, C1-C6 alkoxy, C6-C60 aryl, or C6-C60 heteroaryl.

2. The organic compound according to claim 1, wherein Lₘ is independently selected from any one of a single bond, phenylene, biphenylene, naphthylene, anthrylene, or phenanthrylene.

3. The organic compound according to claim 1, wherein Lₘ is independently selected from any one of a single bond, phenylene, or biphenylene.

4. The organic compound according to claim 1, wherein Lₘ is independently selected from a single bond or any one of the following groups: 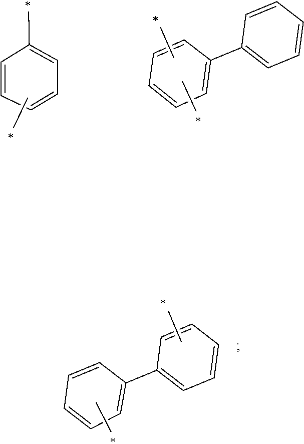
wherein the asterisk denotes a linkage site.

5. The organic compound according to claim 1, wherein Lₘ is independently selected from a single bond or any one of the following groups: 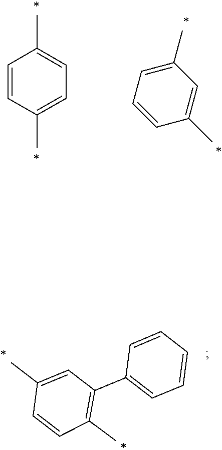
wherein the asterisk denotes a linkage site.

6. The organic compound according to claim 1, wherein n is an integer selected from 1 to 3.

7. The organic compound according to claim 1, wherein Rᵢ is independently selected from any one of substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted 9,10-benzophenanthryl, substituted or unsubstituted pyridyl, substituted or unsubstituted quinolyl, substituted or unsubstituted benzoxazolyl, substituted or unsubstituted carbazolyl, or substituted or unsubstituted arylamino; and
a substituent for substitution is selected from any one of C1-C10 alkyl, C3-C10 cycloalkyl, C1-C6 alkoxy, C6-C60 aryl, or C6-C60 heteroaryl.

8. The organic compound according to claim 1, wherein Rᵢ is independently selected from any one of substituted or unsubstituted phenyl, substituted or unsubstituted biphenyl, substituted or unsubstituted terphenyl, substituted or unsubstituted naphthyl, substituted or unsubstituted 9,10-benzophenanthryl, substituted or unsubstituted pyridyl, substituted or unsubstituted quinolyl, substituted or unsubstituted benzoxazolyl, substituted or unsubstituted carbazolyl, or substituted or unsubstituted arylamino; and
a substituent for substitution is selected from any one of C1-C10 alkyl, C3-C10 cycloalkyl, C1-C6 alkoxy, C6-C60 aryl, or C6-C60 heteroaryl.

9. The organic compound according to claim 1, wherein Rᵢ is independently selected from any one of the following groups: 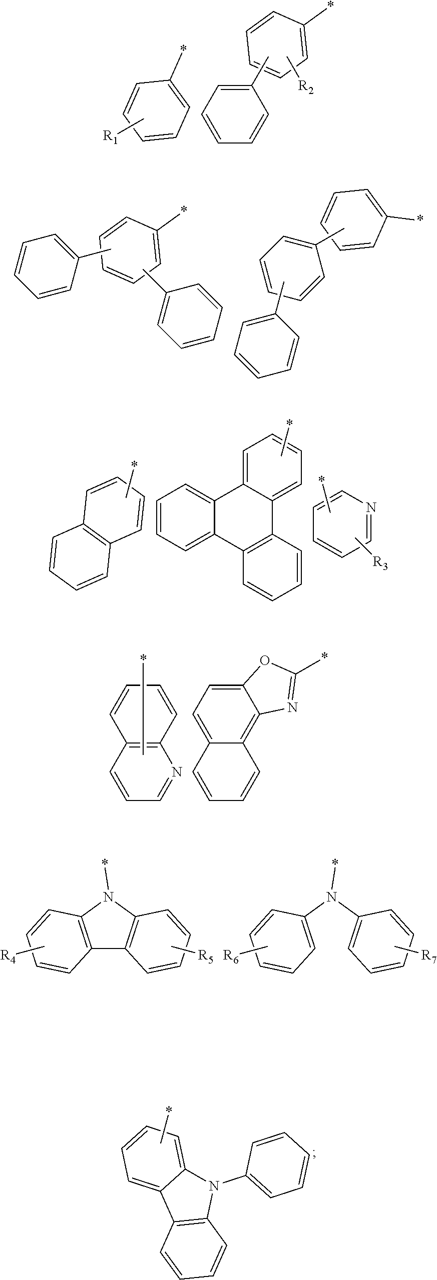
wherein R₁ to R₇ are each independently selected from any one of hydrogen, C1-C10 alkyl, C3-C10 cycloalkyl, C1-C6 alkoxy, C6-C60 aryl, or C6-C60 heteroaryl; and
the asterisk denotes a linkage site.

10. The organic compound according to claim 9, wherein R₁ to R₇ are each independently selected from any one of hydrogen, C1-C10 alkyl, C3-C10 cycloalkyl, or C6-C60 aryl.

11. The organic compound according to claim 9, wherein R₁ and R₂ are each independently selected from any one of hydrogen, methyl, ethyl, propyl, n-butyl, isobutyl, tert-butyl, or cyclohexyl;
R₃ is selected from any one of hydrogen, methyl, ethyl, propyl, or phenyl;
R₄ to R₇ are each independently selected from any one of hydrogen, methyl, ethyl, propyl, n-butyl, isobutyl, tert-butyl, cyclohexyl, or phenyl.

12. The organic compound according to claim 1, wherein Rᵢ is each independently selected from any one of the following groups: 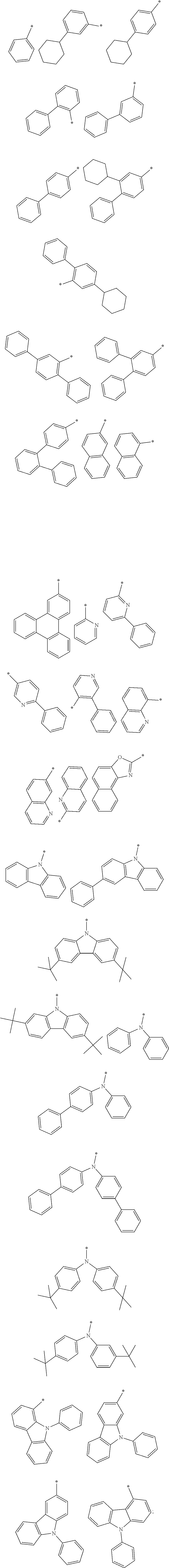
wherein the asterisk denotes a linkage site.

13. The organic compound according to claim 1, wherein j is an integer selected from 1 to 3.

14. The organic compound according to claim 1, wherein R_{c} is selected from H or

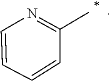

15. The organic compound according to claim 1, wherein the organic compound is selected from any one of the following compounds:

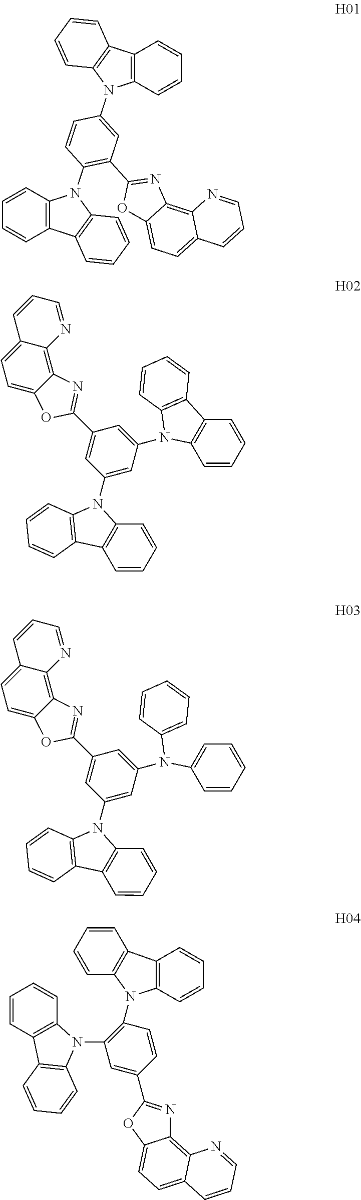

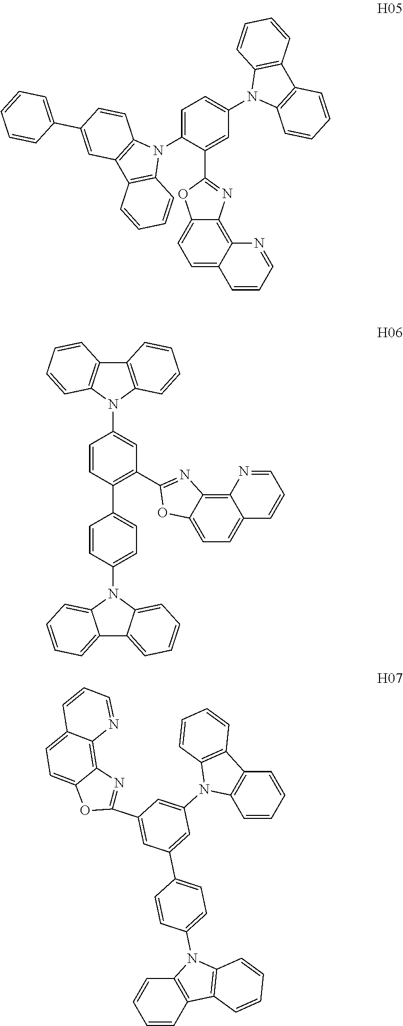

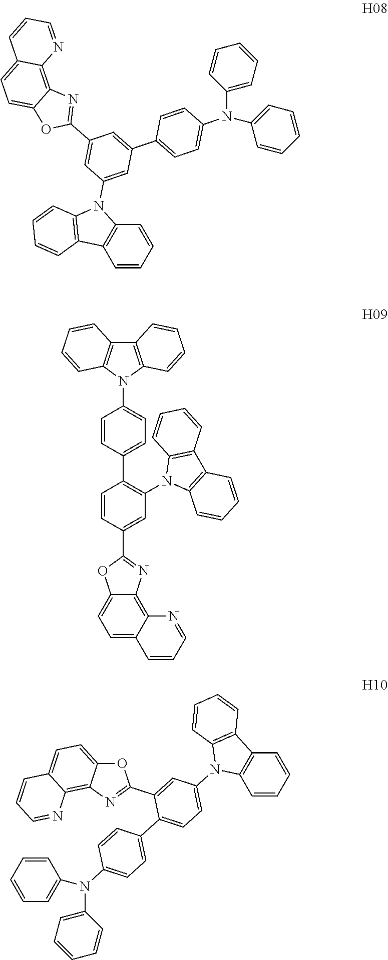 [SMILES: c1ccc(N(c2ccccc2)c2ccc(-c3cc(-c4nc5c(ccc6cccnc65)o4)cc(-n4c5ccccc5c5ccccc54)c3)cc2)cc1 ; c1cnc2c(c1)ccc1oc(-c3ccc(-c4ccc(-n5c6ccccc6c6ccccc65)cc4)c(-n4c5ccccc5c5ccccc54)c3)nc12 ; c1ccc(N(c2ccccc2)c2ccc(-c3ccc(-n4c5ccccc5c5ccccc54)cc3-c3nc4c(ccc5cccnc54)o3)cc2)cc1]

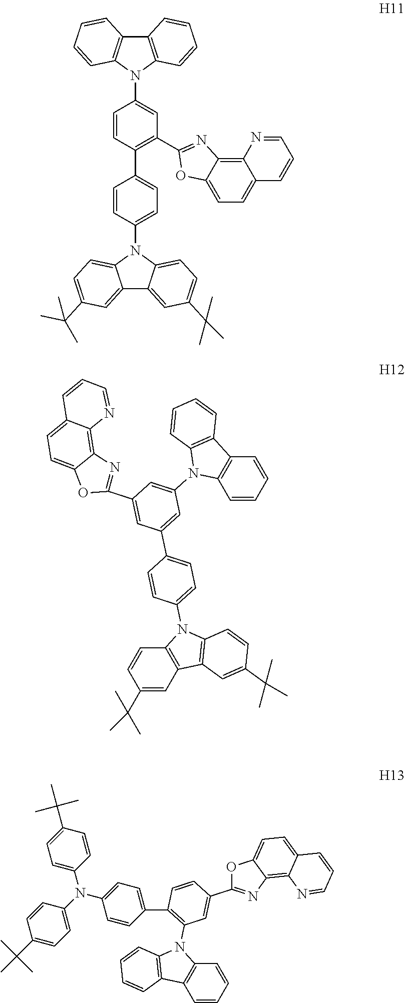

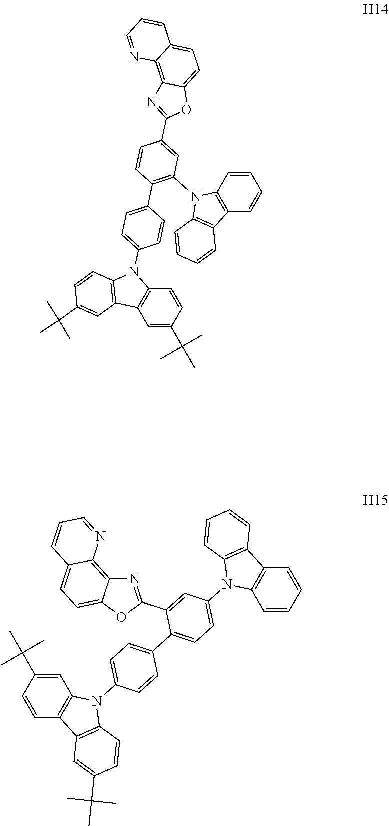 [SMILES: CC(C)(C)c1ccc2c(c1)c1cc(C(C)(C)C)ccc1n2-c1ccc(-c2ccc(-c3nc4c(ccc5cccnc54)o3)cc2-n2c3ccccc3c3ccccc32)cc1 ; CC(C)(C)c1ccc2c(c1)c1ccc(C(C)(C)C)cc1n2-c1ccc(-c2ccc(-n3c4ccccc4c4ccccc43)cc2-c2nc3c(ccc4cccnc43)o2)cc1]

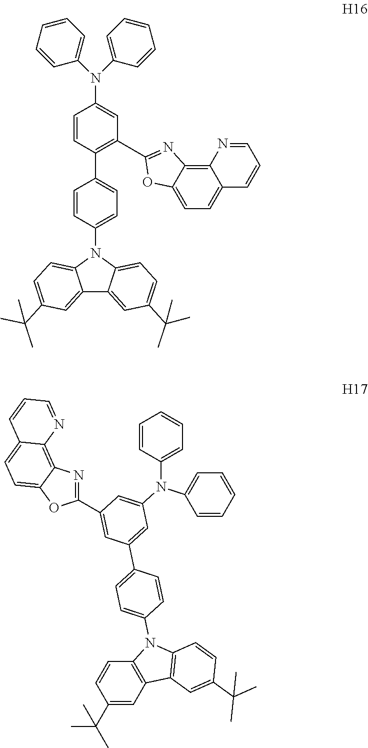 [SMILES: CC(C)(C)c1ccc2c(c1)c1cc(C(C)(C)C)ccc1n2-c1ccc(-c2ccc(N(c3ccccc3)c3ccccc3)cc2-c2nc3c(ccc4cccnc43)o2)cc1 ; CC(C)(C)c1ccc2c(c1)c1cc(C(C)(C)C)ccc1n2-c1ccc(-c2cc(-c3nc4c(ccc5cccnc54)o3)cc(N(c3ccccc3)c3ccccc3)c2)cc1]

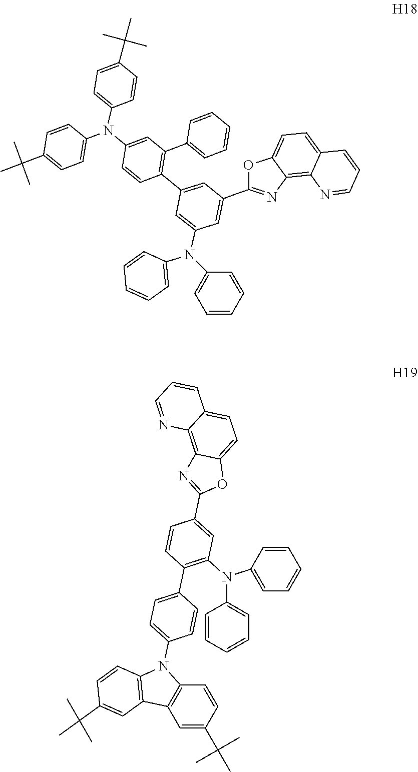

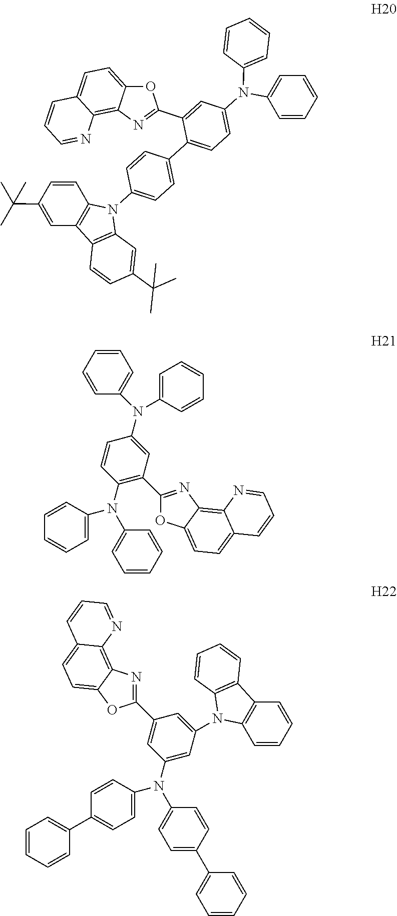 [SMILES: CC(C)(C)c1ccc2c(c1)c1ccc(C(C)(C)C)cc1n2-c1ccc(-c2ccc(N(c3ccccc3)c3ccccc3)cc2-c2nc3c(ccc4cccnc43)o2)cc1 ; c1ccc(N(c2ccccc2)c2ccc(N(c3ccccc3)c3ccccc3)c(-c3nc4c(ccc5cccnc54)o3)c2)cc1 ; c1ccc(-c2ccc(N(c3ccc(-c4ccccc4)cc3)c3cc(-c4nc5c(ccc6cccnc65)o4)cc(-n4c5ccccc5c5ccccc54)c3)cc2)cc1]

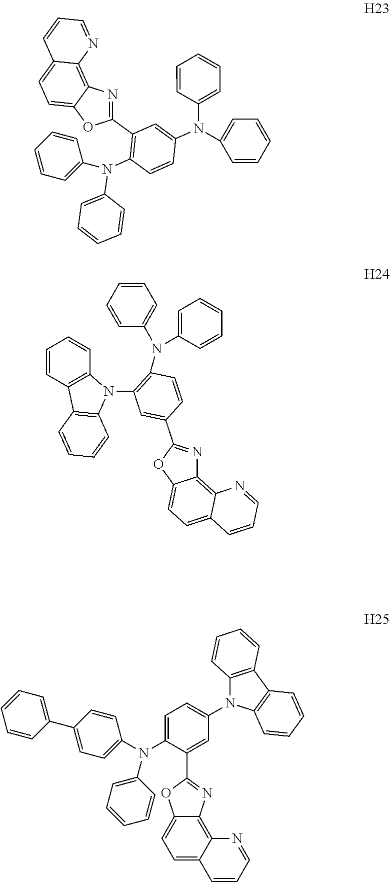 [SMILES: c1ccc(N(c2ccccc2)c2ccc(N(c3ccccc3)c3ccccc3)c(-c3nc4c(ccc5cccnc54)o3)c2)cc1 ; c1ccc(N(c2ccccc2)c2ccc(-c3nc4c(ccc5cccnc54)o3)cc2-n2c3ccccc3c3ccccc32)cc1 ; c1ccc(-c2ccc(N(c3ccccc3)c3ccc(-n4c5ccccc5c5ccccc54)cc3-c3nc4c(ccc5cccnc54)o3)cc2)cc1]

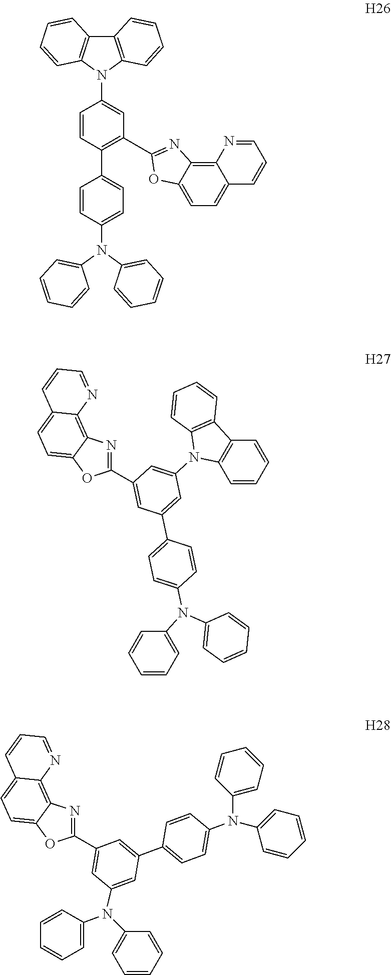 [SMILES: c1ccc(N(c2ccccc2)c2ccc(-c3ccc(-n4c5ccccc5c5ccccc54)cc3-c3nc4c(ccc5cccnc54)o3)cc2)cc1 ; c1ccc(N(c2ccccc2)c2ccc(-c3cc(-c4nc5c(ccc6cccnc65)o4)cc(-n4c5ccccc5c5ccccc54)c3)cc2)cc1 ; c1ccc(N(c2ccccc2)c2ccc(-c3cc(-c4nc5c(ccc6cccnc65)o4)cc(N(c4ccccc4)c4ccccc4)c3)cc2)cc1]

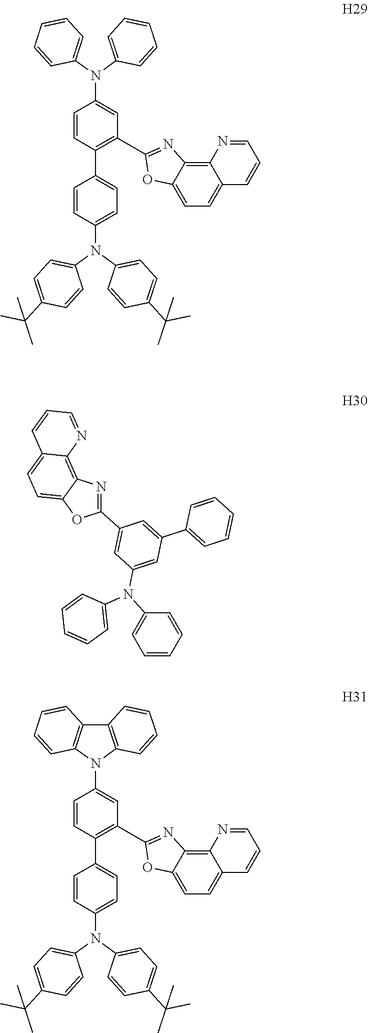 [SMILES: CC(C)(C)c1ccc(N(c2ccc(-c3ccc(N(c4ccccc4)c4ccccc4)cc3-c3nc4c(ccc5cccnc54)o3)cc2)c2ccc(C(C)(C)C)cc2)cc1 ; c1ccc(-c2cc(-c3nc4c(ccc5cccnc54)o3)cc(N(c3ccccc3)c3ccccc3)c2)cc1 ; CC(C)(C)c1ccc(N(c2ccc(-c3ccc(-n4c5ccccc5c5ccccc54)cc3-c3nc4c(ccc5cccnc54)o3)cc2)c2ccc(C(C)(C)C)cc2)cc1]

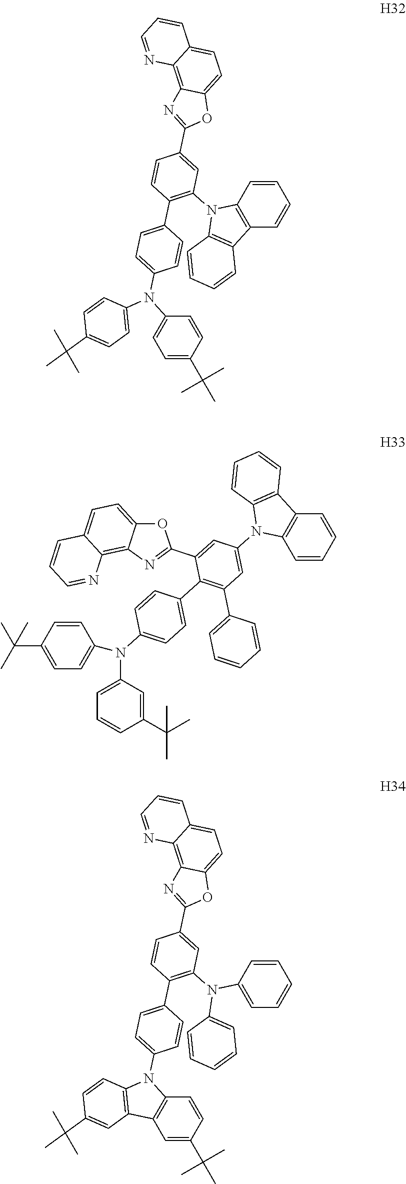

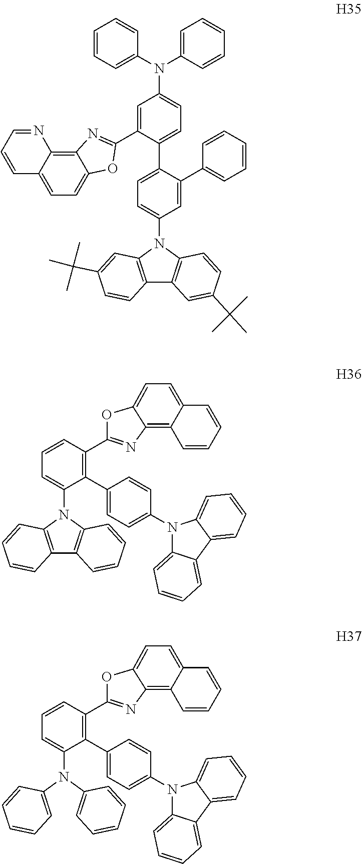 [SMILES: CC(C)(C)c1ccc2c(c1)c1ccc(C(C)(C)C)cc1n2-c1ccc(-c2ccc(N(c3ccccc3)c3ccccc3)cc2-c2nc3c(ccc4cccnc43)o2)c(-c2ccccc2)c1 ; c1cc(-c2nc3c(ccc4ccccc43)o2)c(-c2ccc(-n3c4ccccc4c4ccccc43)cc2)c(-n2c3ccccc3c3ccccc32)c1 ; c1ccc(N(c2ccccc2)c2cccc(-c3nc4c(ccc5ccccc54)o3)c2-c2ccc(-n3c4ccccc4c4ccccc43)cc2)cc1]

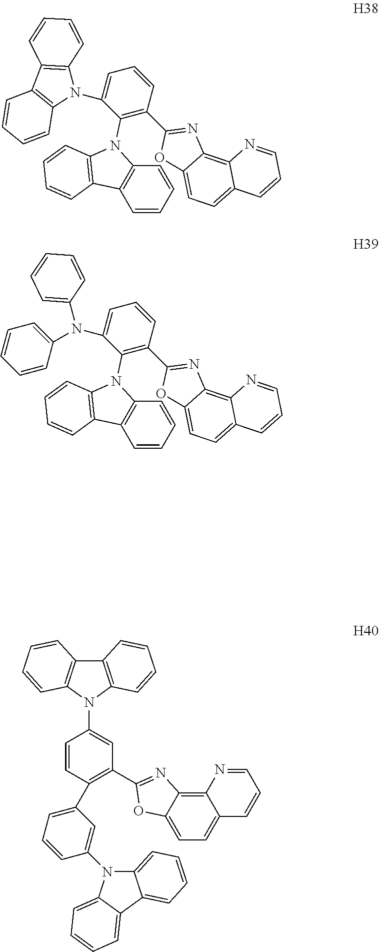 [SMILES: c1cc(-c2nc3c(ccc4cccnc43)o2)c(-n2c3ccccc3c3ccccc32)c(-n2c3ccccc3c3ccccc32)c1 ; c1ccc(N(c2ccccc2)c2cccc(-c3nc4c(ccc5cccnc54)o3)c2-n2c3ccccc3c3ccccc32)cc1 ; c1cc(-c2ccc(-n3c4ccccc4c4ccccc43)cc2-c2nc3c(ccc4cccnc43)o2)cc(-n2c3ccccc3c3ccccc32)c1]

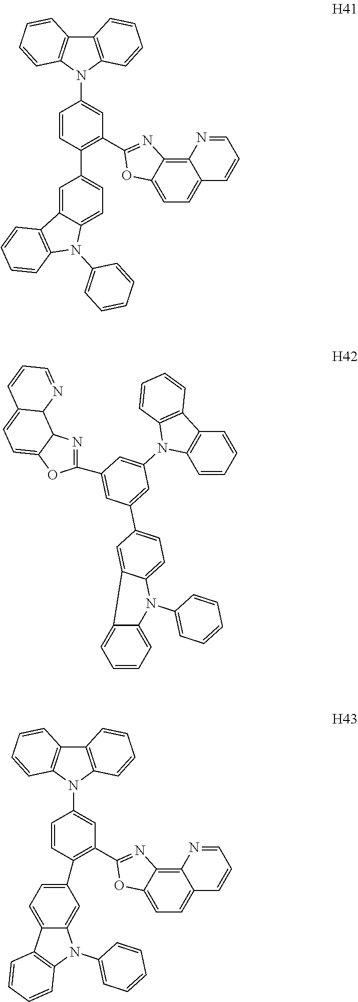 [SMILES: c1ccc(-n2c3ccccc3c3cc(-c4ccc(-n5c6ccccc6c6ccccc65)cc4-c4nc5c(ccc6cccnc65)o4)ccc32)cc1 ; C1=CC2=CC=C3OC(c4cc(-c5ccc6c(c5)c5ccccc5n6-c5ccccc5)cc(-n5c6ccccc6c6ccccc65)c4)=NC3C2N=C1 ; c1ccc(-n2c3ccccc3c3ccc(-c4ccc(-n5c6ccccc6c6ccccc65)cc4-c4nc5c(ccc6cccnc65)o4)cc32)cc1]

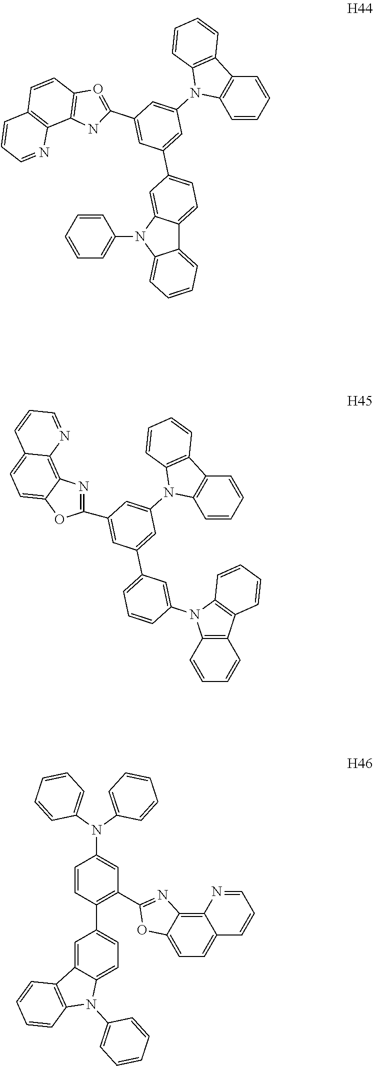

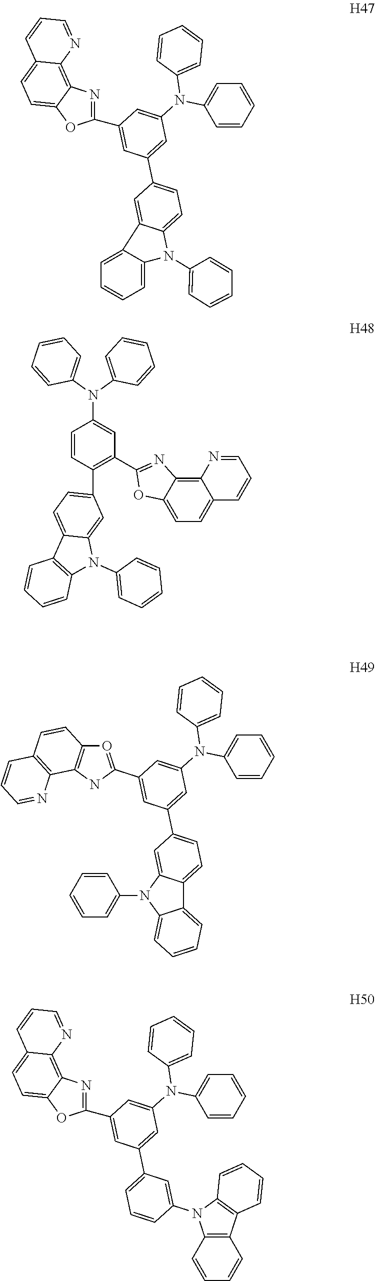

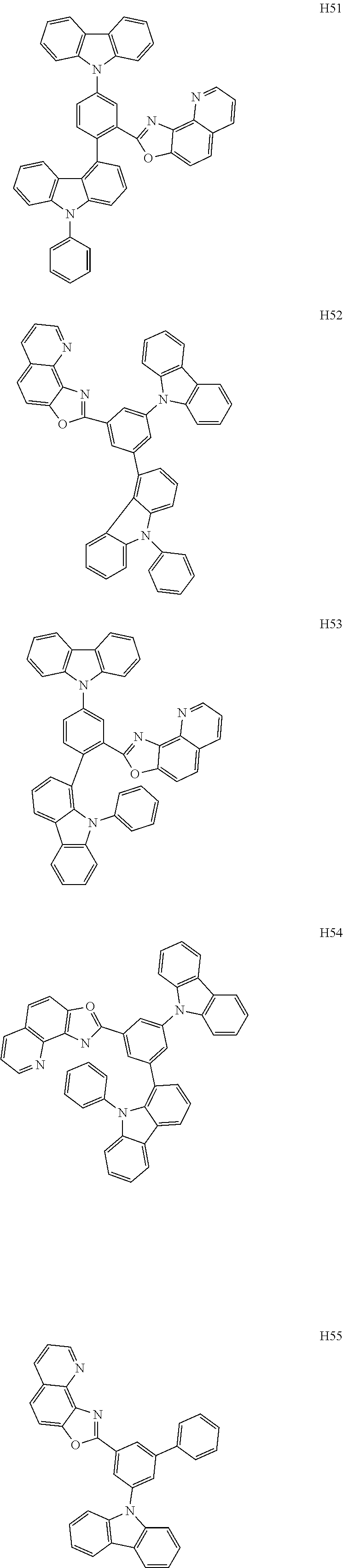

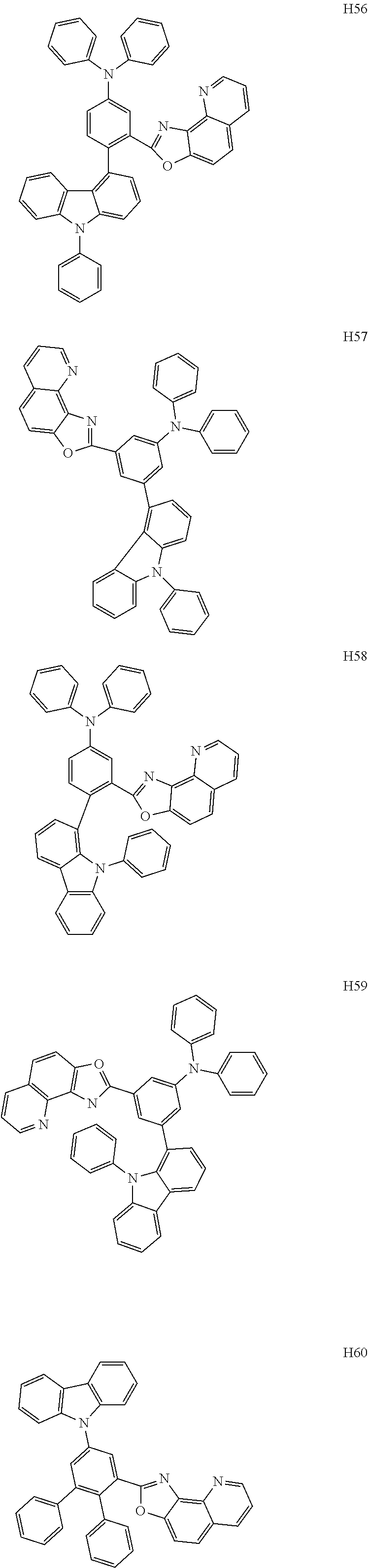

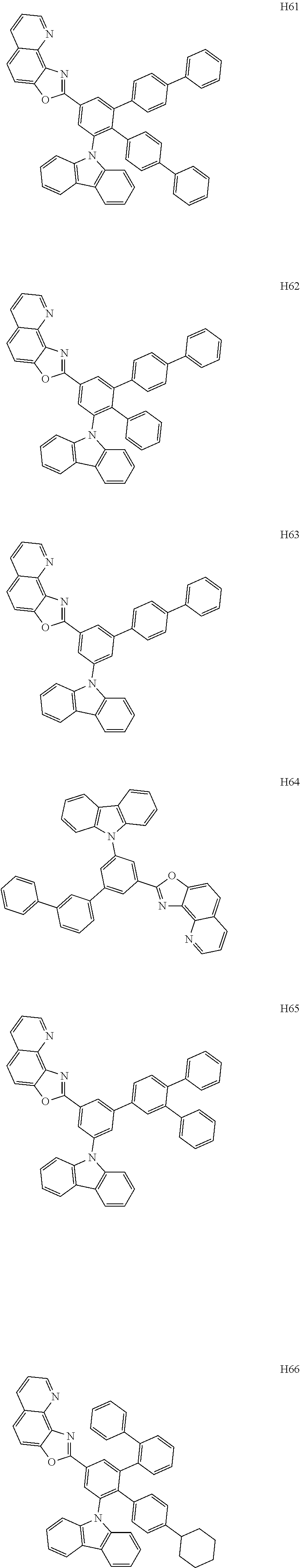

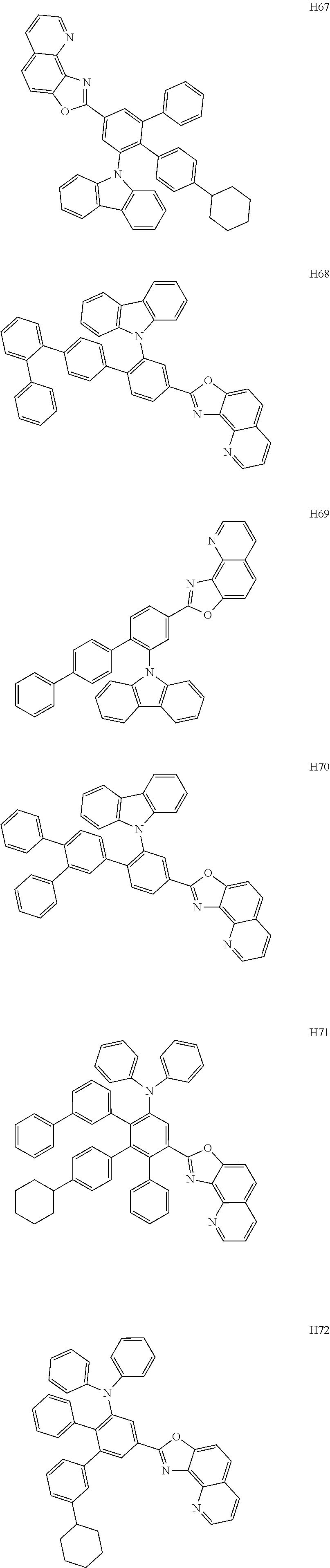

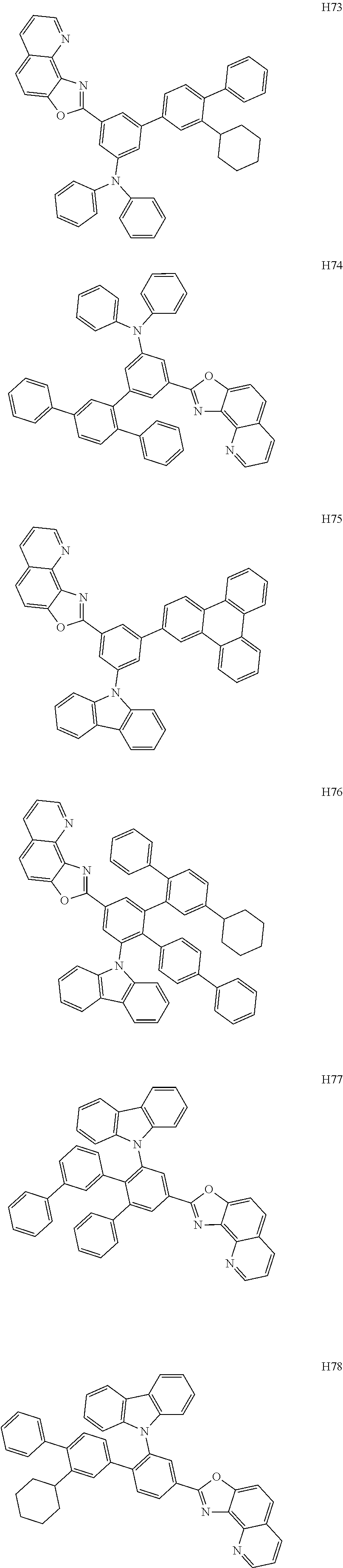

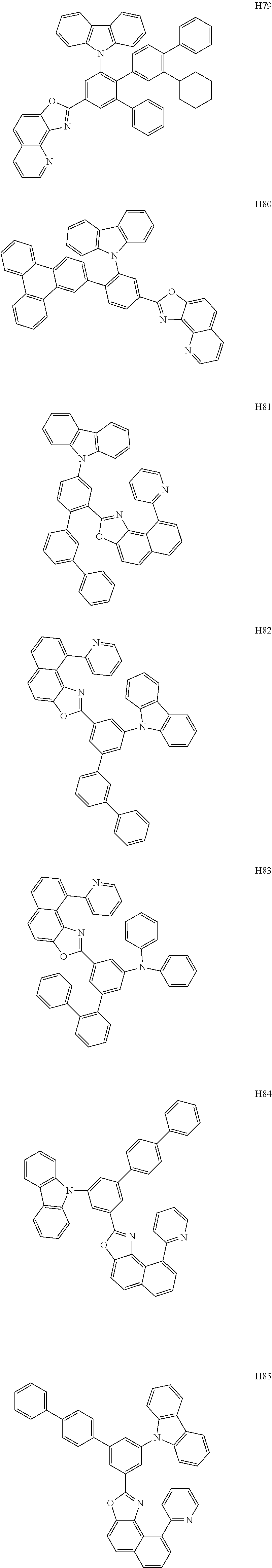

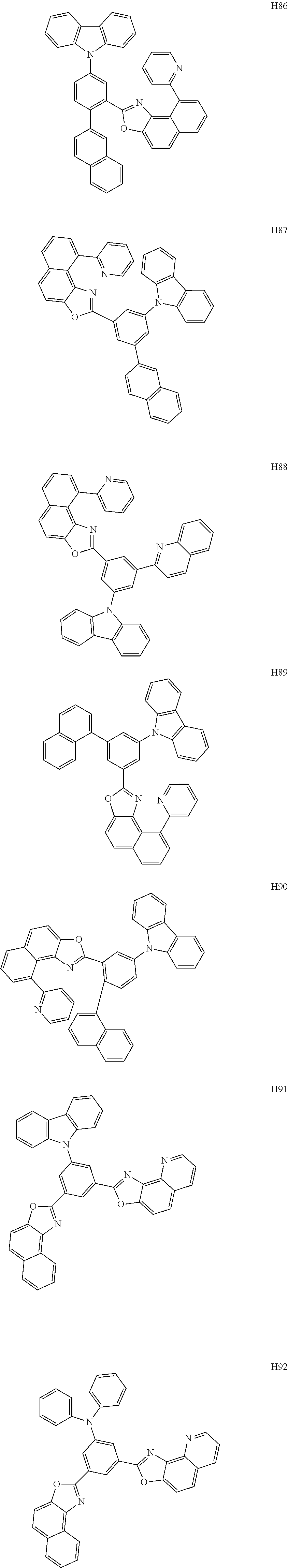

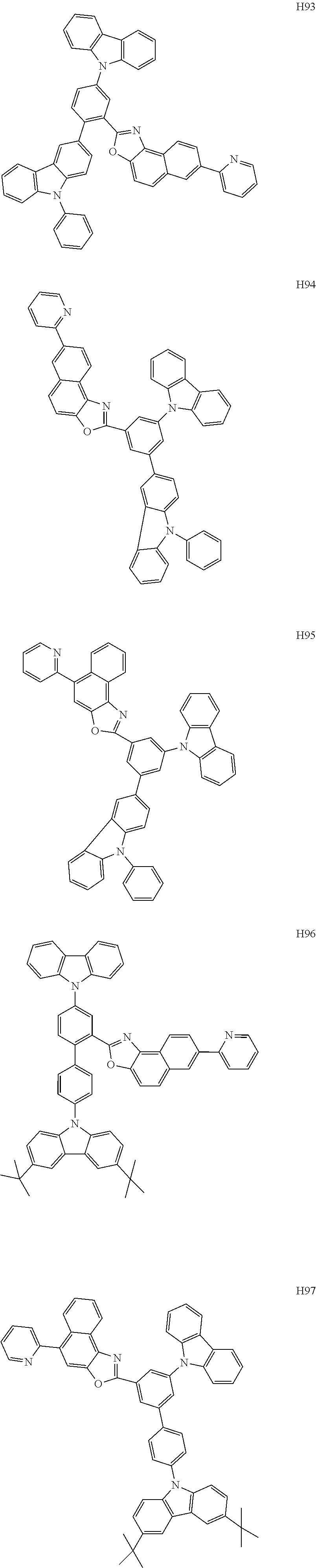

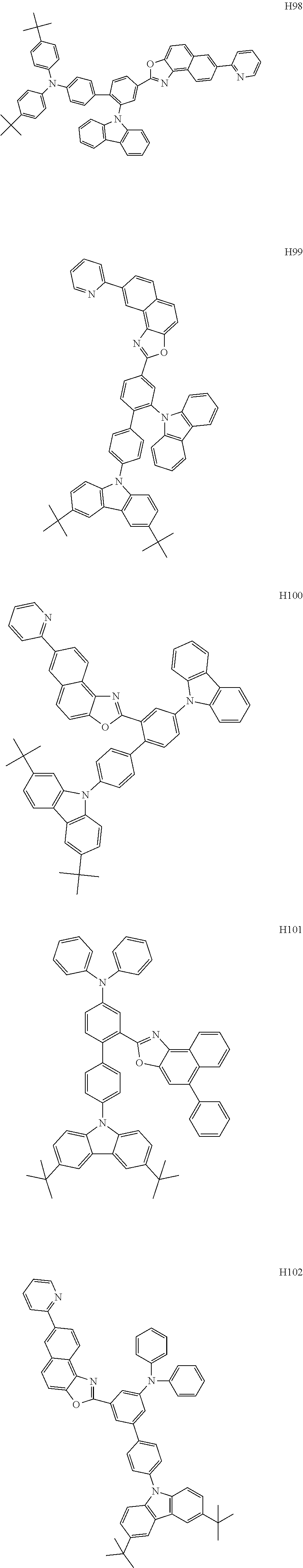

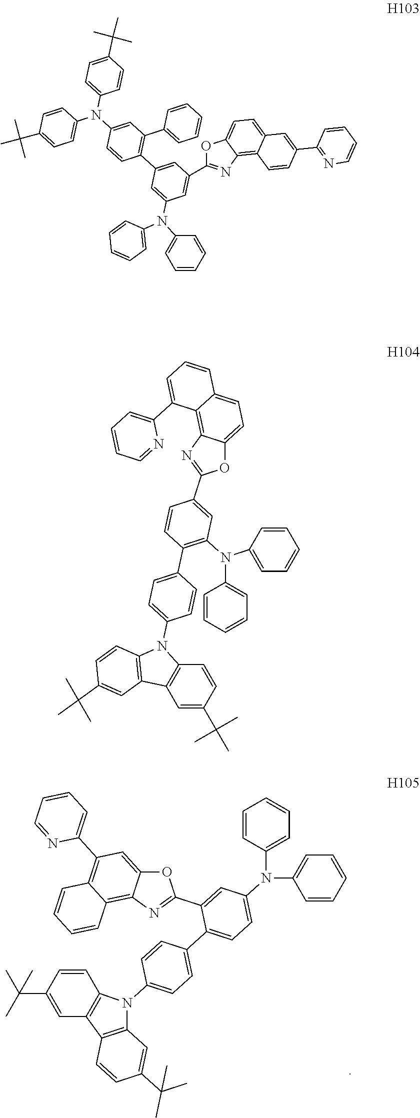 [SMILES: CC(C)(C)c1ccc(N(c2ccc(C(C)(C)C)cc2)c2ccc(-c3cc(-c4nc5c(ccc6cc(-c7ccccn7)ccc65)o4)cc(N(c4ccccc4)c4ccccc4)c3)c(-c3ccccc3)c2)cc1 ; CC(C)(C)c1ccc2c(c1)c1cc(C(C)(C)C)ccc1n2-c1ccc(-c2ccc(-c3nc4c(ccc5cccc(-c6ccccn6)c54)o3)cc2N(c2ccccc2)c2ccccc2)cc1 ; CC(C)(C)c1ccc2c(c1)c1ccc(C(C)(C)C)cc1n2-c1ccc(-c2ccc(N(c3ccccc3)c3ccccc3)cc2-c2nc3c(cc(-c4ccccn4)c4ccccc43)o2)cc1]

16. An OLED display panel, comprising a first electrode, a second electrode, and an organic thin film layer provided between the first electrode and the second electrode;
wherein the organic thin film layer comprises a light-emitting layer; and
a host material of the light-emitting layer comprises an organic compound;
wherein the organic compound has a structure represented by Formula (I): 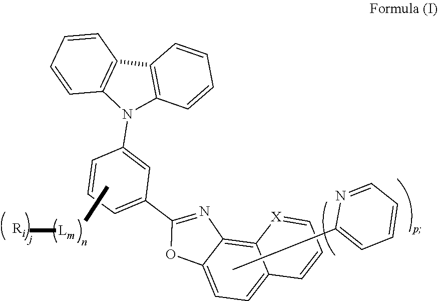
wherein Lₘ is independently selected from any one of a single bond, substituted or unsubstituted C6-C40 arylene, or substituted or unsubstituted C5-C40 heteroarylene;
n is an integer selected from 1 to 4, and m is an integer selected from 1 to n;
Rᵢ is independently selected from any one of substituted or unsubstituted C6-C60 aryl, substituted or unsubstituted C6-C60 heteroaryl, substituted or unsubstituted C6-C60 fused ring aryl, or substituted or unsubstituted C6-C30 arylamino;
j is an integer selected from 1 to 4, and i is an integer selected from 1 to j; and j and n have the same value;
X is selected from N or C—R_{c}; wherein R_{c} is selected from H or 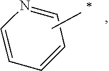
and the asterisk denotes a linkage site;
p is an integer selected from 0 to 5;
in formula (I), the dashed line denotes a single bond or nothing exists; and
a substituent for substitution is selected from any one of C1-C10 alkyl, C3-C10 cycloalkyl, C1-C6 alkoxy, C6-C60 aryl, or C6-C60 heteroaryl.

17. The OLED display panel according to claim 16, wherein the organic thin film layer further comprises any one or a combination of at least two of a hole injection layer, a hole transport layer, an electron blocking layer, a hole blocking layer, an electron transport layer, or an electron injection layer.

18. An electronic device, comprising an OLED display panel;
wherein the OLED display panel comprises a first electrode, a second electrode, and an organic thin film layer provided between the first electrode and the second electrode;
wherein the organic thin film layer comprises a light-emitting layer; and
a host material of the light-emitting layer comprises an organic compound;
wherein the organic compound has a structure represented by Formula (I): 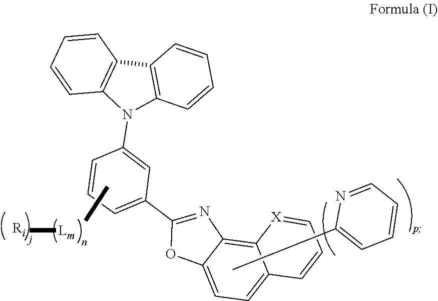
wherein Lₘ is independently selected from any one of a single bond, substituted or unsubstituted C6-C40 arylene, or substituted or unsubstituted C5-C40 heteroarylene;
n is an integer selected from 1 to 4, and m is an integer selected from 1 to n;
Rᵢ is independently selected from any one of substituted or unsubstituted C6-C60 aryl, substituted or unsubstituted C6-C60 heteroaryl, substituted or unsubstituted C6-C60 fused ring aryl, or substituted or unsubstituted C6-C30 arylamino;
j is an integer selected from 1 to 4, and i is an integer selected from 1 to j; and j and n have the same value;
X is selected from N or C—R_{c}; wherein R_{c} is selected from H or 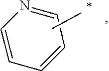
and the asterisk denotes a linkage site;
p is an integer selected from 0 to 5;
in formula (I), the dashed line denotes a single bond or nothing exists; and
a substituent for substitution is selected from any one of C1-C10 alkyl, C3-C10 cycloalkyl, C1-C6 alkoxy, C6-C60 aryl, or C6-C60 heteroaryl.
